(19) United States
(12) Patent Application Publication
(10) Pub. No.: US 2025/0125054 A1
(43) Pub. Date: Apr. 17, 2025

(54) SYSTEMS AND METHODS FOR IDENTIFYING PROSTATE CANCER PATIENTS AT HIGH-RISK OF PROGRESSION

(71) Applicants: PATHOMIQ INC., Saratoga, CA (US); Icahn School of Medicine at Mount Sinai, New York, NY (US)

(72) Inventors: Hassan Muhammad, Newburgh, NY (US); Chensu Xie, Bothell, WA (US); Parag Jain, Palo Alto, CA (US); Rajat Roy, Saratoga, CA (US); Ashutosh K. Tewari, New York, NY (US); Dimple Chakravarty, Oradell, NJ (US); Sujit S. Nair, Rego Park, NY (US)

(21) Appl. No.: 18/918,891

(22) Filed: Oct. 17, 2024

Related U.S. Application Data
(60) Provisional application No. 63/544,562, filed on Oct. 17, 2023.

(51) Int. Cl. G16H 50/20; A61B 5/00; G06T 7/00; G16B 20/00; G16H 10/40

(52) U.S. Cl. CPC G16H 50/20; A61B 5/4381; G06T 7/0012; G16B 20/00; G16H 10/40; G06T 2207/20021; G06T 2207/20036; G06T 2207/20081; G06T 2207/20084; G06T 2207/30024; G06T 2207/30081

(57) ABSTRACT

Disclosed are systems and methods for identifying prostate cancer patients at high-risk of progression among clinically intermediate risk group. Images of patient cells are obtained and tiled into subsets of smaller images. Using a trained machine learning model, a morphology quantification process is performed on the subsets of smaller images. Portions of the images are input into the trained machine learning models. The trained machine learning model determines the occurrence of likely cancer cells and classifies the cancer cells with a grading. The system then uses this out of the machine learning model to identify whether the cells in the subsets of smaller images indicate whether a prostate cancer patient is at a risk of progression among a clinically intermediate risk group.

### RELATED APPLICATIONS

This application claims the benefit of priority to U.S. Provisional Application No. 63/544,562, filed on Oct. 17, 2023, which is hereby incorporated by reference in its entirety.

### FIELD

This invention relates generally to the field of disease detection and treatment identification, and in particular to using artificial intelligence in identifying prostate cancer patients at high-risk of progression among clinically intermediate risk group.

### BACKGROUND

Prostate cancer (PCa) is the second most diagnosed cancer among US men. About 70% of PCa patients are either cured after the first-line of intervention (radical prostatectomy and/or ionizing radiation therapy) or their disease remains organ-confined for life. Thus, the treatment decision for PCa relies on a patient's likelihood of disease progression. Patients whose prostate has been debulked with the first line of therapy are generally followed by a simple blood test to detect prostate specific antigen (PSA) levels in the blood. About 30% of the patients with rising blood PSA after the first line of treatment, defined as biochemical relapse (BCR), are considered to be at high risk of having progressing metastatic PCa and need aggressive treatment.

Generally, methods such as Gleason grading (GG), tumor volume measurement, and clinical staging (TNM) are used in the clinic to prognosticate the risk of disease progression. About 60% of the patients, who do not exhibit explicit high or low risk characteristics when assessed using these methods are considered intermediate-risk and do not have a clearly defined treatment plan. Roughly half of the patients within this group return to the clinic with disease progression after their first line of therapy. While these intermediate-risk patients are further classified into Gleason Grade Group 2 and 3 ((GG) 3+4 and 4+3, respectively), studies indicate that a large number of patients with progressing PCa can be found in both groups.

The application of more aggressive therapies at the earlier stages of PCa for patients with high risk of progression have improved patient outcomes. For example, the clinical trials PROSPER, SPARTAN, ARAMIS, added enzalutamide, apalutamide, or darolutamide, respectively, to standard androgen deprivation therapy (ADT) to both metastatic and non-metastatic castrate resistant PCa (CRPC). All of the trials showed improvements in multiple clinical end points such as overall survival, time to metastasis, and time to biochemical recurrence. Thus, it is important to clearly and efficiently identify intermediate risk patients with high risk of progression at an earlier stage of PCa, to benefit from timely intervention with adjuvant therapies after the first line of therapy in order to improve overall disease outcome.

In general, the best indicators of risk of BCR are GG and TNM staging. However, recently some clinicians are using more advanced methods such as genomic tests. Based on the results of the whole tissue genomic analysis, a few sets of genomic markers included in the scoring systems of DECIPHER, POLARIS, Oncotype GPS, etc. have been identified for PCa prognosis and have recently started entering clinical practice. Most of these genome-based scores, however, marginally improved the prognostic accuracy of GGS thus far. Their diagnostic accuracy (at <70% for the intermediate risk group patients), requirement of a relatively large volume of tissue samples often unachievable in prostate biopsies, high pricing, and long turn-around time pose significant hurdle in clinical applications.

In recent years, computer vision-based deep learning has shown to recognize objects and diagnose diseases from histopathology whole slide images (WSIs) with impressive accuracy. Prior in silico studies that we and other have performed have shown deep learning models with performance at par with human experts for diagnostic tasks such as tumor detection and grading. The models deliver accurate detection and quantification of known histological patterns of established clinical significance and reduce inter-observer and intra-observer variability among general and subspecialty expert pathologists. In parallel, methods focusing on directly learning morphological features that are associated with clinical outcome have been developed. Some success in this field includes predicting overall survival from colorectal cancer using histology slides and immune response in lung cancer treatment. Our prior work has shown ability to predict response to neoadjuvant chemotherapy in triple negative breast cancer.

Similarly, in the area of PCa, various AI-enabled digital pathology-based methods have also been published, citing potential to improve patient risk stratification by improving GG using automated quantification or, in some cases, through novel biomarker discovery. Methods which take the route of quantifying GG, however, have some weaknesses. Distinguishing within GG 7 (3+4 and 4+3) have still vexed many algorithms. One study merged these two groups together into one category for prediction, defeating the purpose of the current GGG stratification. Another study was able to predict GG adequately overall but also struggled in distinguishing 3+4 and 4+3. Both methods are limited to predicting disease progression to how well they can quantify morphologies based on GG. These limitations of GG indicate that there are further markers to be discovered within the prostate tumor landscape for better patient stratification to guide successful clinical intervention.

Very recently, an AI-based retrospective tissue image analysis of a large RTOG trial prostate tissue images identified intermediate-risk patients who will benefit from adding short term androgen deprivation therapy (ADT) with ionizing radiation [19, 20]. No such stratification has been available for intermediate-risk patients about the benefits of radical prostatectomy (RP) and subsequent ADT and/or anti-androgen therapy. This method showed the power of combining self-supervised learning to learn high dimensional features from WSIs with standard clinical features to provide a risk of relapse at 5 years with an AUC of 0.67. Another study extracted gland features in PCa WSIs, independent of GG patterns, in order to rank patients by risk of BCR. Their method produced a concordance index of 0.68 on a test set. This model, however, was limited to hand-engineered morphological features. While these features are quantitative, they are still limited to human perception and thus, suffers from subjectivity.

Consequently, there is a need for computer aided systems and methods that can identifying prostate cancer patients at high-risk of progression among clinically intermediate risk group.

### SUMMARY

The systems and methods described herein provide an AI-powered platform that objectively extract features at visual and more importantly at sub-visual levels and accurately identified all GG patterns, including distinguishing 3+4 and 4+3, with a weighted kappa score of 98%. To bridge this advanced AI-powered method with clinically relevant biological significance, we performed genomic analysis in areas of tissue indicated as especially high, and determined a potentially new STING pathway-related PCa biomarker, TMEM173. Through a combination of semi-supervised learning and accurate morphological quantification, we developed a system which only relies on digitized H&E WSIs to predict risk of BCR within 36 months and metastatic disease more accurately than GG, TNM, standard nomograms, as well as genomic tests. The system delivers a risk score using a cost-efficient, secure, and quick algorithm which preserves the WSIs analyzed, overcoming many hurdles of genomic analysis such as paucity of material, shipping of tissue samples, and others listed above.

### BRIEF DESCRIPTION OF THE DRAWINGS

These drawings and the associated description herein are provided to illustrate specific embodiments of the invention and are not intended to be limiting.

FIG. 1 illustrates an example of disease progression stages in relation to available treatment options.

FIG. 2 illustrates a patient outcome prediction system according to an embodiment.

FIG. 3 illustrates a disease detection and grading module (DDGM) and the components therein according to an embodiment.

FIG. 4 illustrates an example implementation and operation of a vectorization module.

FIG. 5 illustrates a sub-morphology detector (SMD) according to an embodiment.

FIG. 6 illustrates an example operation of the SMD according to an embodiment.

FIG. 7 illustrates a ROI and outcome prediction module (ROPM) and the components therein according to an embodiment.

FIG. 8 illustrates a flowchart of a method of the operations of the ROI and outcome prediction module (ROPM) according to an embodiment.

FIG. 9 illustrates a tissue slide that has been processed through the patient outcome prediction system.

FIG. 10 illustrates an application of proteo-genomic technique to regions of interest identified by ROPM to determine proteins or DNA/RNA markers that correlate with patient outcome.

FIG. 11 illustrates an H&E image slide annotated with patch-level morphological patterns, using the described embodiments.

FIG. 12 illustrates a diagram of data usage in some embodiments.

FIG. 13 illustrates a table of training and test data.

FIG. 14 illustrates an embodiment of processing patient slide data and determining a final output of risk prediction.

FIG. 15 illustrates a flowchart of an embodiment of a method performed by the system.

FIG. 16 illustrates a table describing overall performance of the system's prediction performance.

FIG. 17 illustrates a chart describing risk or BCR and risk of MET.

FIG. 18 illustrates a chart describing hazard ratios.

FIG. 19 illustrates the system substratifying GGG2 and GGG3 patients with high statistical significance.

FIG. 20 illustrates the tables describing risk stratification and decision curve analysis.

FIG. 21 illustrates an embodiment of the system (PATHOMIQ_PRAD) processing pipeline.

FIG. 22 illustrates a high-level cluster map of various prostate cancer morphologies learned by the system.

FIG. 23 illustrates examples of high- and low-scoring morphological areas of a tumor microenvironment as determined by the system.

FIG. 24 illustrates an example of a processing workflow for prostate cancer WSI analysis and validation according to an embodiment of the system.

FIG. 25 illustrates a table describing predictive accuracy in terms of a concordance index.

FIG. 26 illustrates a table describing predictive accuracy.

FIG. 27 illustrates a table describing univariate analysis results.

FIG. 28 illustrates a table describing multivariate analysis results.

FIG. 29 illustrates a table describing univariate analysis results.

FIG. 30 illustrates a table describing multivariate analysis results.

FIG. 31 is a diagram illustrating an exemplary computer that may perform processing in some embodiments.

### DETAILED DESCRIPTION

The following detailed description of certain embodiments presents various descriptions of specific embodiments of the invention. However, the invention can be embodied in a multitude of different ways as defined and covered by the claims.

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

When the terms “one”, “a” or “an” are used in the disclosure, they mean “at least one” or “one or more”, unless otherwise indicated.

In some embodiments, the systems and methods described herein provides a novel and previously “unknown” morphological features on digitized images of H&E-stained slides that drive tumor progression. The system converts image patches into mathematical vector representations to generate hundreds of clusters of morphologically similar patterns using state-of-the-art Deep Convolutional Neural Network (CNN)-based models. It then ranks these image clusters to identify novel Regions of Interest (ROIs) on the H&E-stained slides which have high or low prognostic/predictive values correlated to patient disease outcome information to generate a morphometric score for therapy response prediction. These ROIs capture both cancer as well as TME landscapes, including spatial distribution of the immune and stromal cells relative to the cancer zones that play major roles in tumor progression and resistance to therapy.

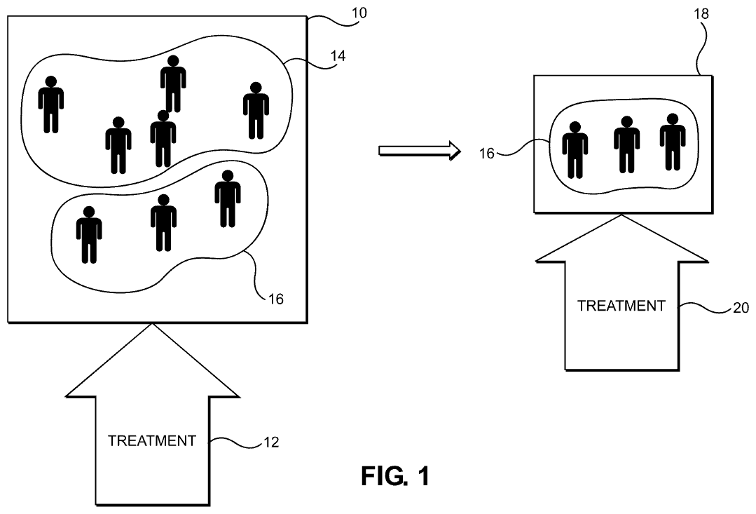

In the context of disease treatment, current medical practice and standard-of-care (SOC) might treat patients based on stages of a disease and the patient's responsiveness to available treatments for those stages. These treatments in case of cancer may include administrating drugs, radiation therapy, surgery or other forms of treatment. If the patient does not respond to available treatments for the stage of disease the patient is in, the patient transitions to a different stage, where different, and potentially more aggressive treatment options may be applied. Later stage treatment options may include experimental or advanced therapy options. FIG. 1 illustrates an example of disease progression stages in relation to available treatment options. In the first disease stage 10, treatment options 12 are applied. A first group of patients 14 respond well to the treatment options 12. A second group of patients 16 do not respond well to the treatment options 12 and their disease progresses to the disease stage 18. Treatment options 20 are available and may be applied for the second group patients 16 in the disease stage 18.

The dynamics of disease progression and applied treatment, in some cases, are as follows. Most patients in earlier stages, respond well to treatment, but a smaller percentage of patients in earlier stages do not respond well to the treatment options applied in those earlier stages. For example, the first group of patients 14 that respond well to treatment options 12 can be a substantially larger number than the second patient group 16 that do not respond well to the treatment options 12. Furthermore, the treatment options that are available to be applied in the later stages can have more efficacy if they are applied in earlier stages. In other words, for the second patient group 16, if treatment options 20, were applied, when those patients were in an earlier disease stage 10, the treatment options 20 may have had more efficacy.

Furthermore, later stage treatment options 20 can include potentially more aggressive treatments or experimental advance therapy options. In some cases, the late stage treatment options 20 can be experimental in nature and can include treatment options that governmental approval may not yet have been obtained. Nonetheless, the second group patients 16 may substantially benefit from those treatments if they were applied in an earlier disease stage 10. Consequently, in terms of disease treatment efficacy and treatment discovery, systems and methods that help early identification of patients that respond well to available treatment options can be beneficial and needed.

Also, pharmaceutical companies are running several thousand clinical trials to get advanced and novel drugs to market across several cancer indications. Lack of reliable predictive biomarkers to identify responders vs non-responders to these drugs result in random selection of patients for the trials and contributes to the low success rate. Even when some of these trials succeed, only a small percentage of patients respond to the drugs when administered in clinical practice. Consequently, there is a huge unmet need for pharmaceutical companies to identify responders to a new drug early on.

Biochemical signatures, biomarkers etc. can be used to predict patient outcome. Some genomics and proteomics techniques to discover biomarkers that predict patient response are focused on biochemical markers and the structured molecular data of those biochemical markers, such as DNA, RNA, and protein data. This approach has major challenges. First, molecular analysis is done from DNA, RNA, and protein extracted from whole tissue which delivers an average molecular signature across tens of thousands of cancer, benign and micro-environmental (e.g., stroma, immune etc.) cells. Consequently, this approach works better when a single or few genes are heavily overexpressed or under-expressed across an entire tissue. However, tumors can be inherently heterogeneous, and there are several molecular subtypes with varying levels of aggressiveness that show up in the same tumor and the tumor micro-environment. This molecular signal gets lost when averaged over an entire tissue.

Second, biochemical, molecular, structural or other analysis of tumor alone do not present a full picture of the disease. In many cases, it is the spatial interaction of the tumor with the tumor micro-environment (TME), including the stroma, several types of immune cells, blood vessels etc., whose interplay determines patient response. Many current genomic analyses are not able to capture the TME dynamics, nor is there one single RNA or protein that can be linked to driving patient response. Nevertheless, Histopathology remains the cornerstone of cancer diagnosis. Many molecular changes and TME elements that are linked to disease can result in morphological changes that are visible on tissue slides. Consequently, systems and methods that can identify and extract morphometric features that correlate with patient outcome from histopathology slides are valuable in disease treatment. Therefore, it is advantageous to employ artificial intelligence (AI) in an unsupervised manner to identify and extract these morphologic features.

Furthermore, the field of studying biomarkers and identification of morphologic features for drug and treatment discovery can be slowed down by the sheer number of samples and patient data that need to be analyzed to identify biomarkers of interest. For example, some methods rely on or work in conjunction with laboratory test results. Described embodiments substantially reduce the volume and number of data that need to be analyzed in a laboratory environment, making the applications of the described embodiments more practical than existing systems. For example, the described embodiments can identify regions of interest (ROIs) on tissue slides that are more predictive, and more promising or relevant for performing laboratory molecular analysis to identify predictive biomarkers of patient response. The identification of aberrant genes/proteins present in the ROI known to be involved in therapy response prediction may also enhance easy detection of disease or therapy response biomarkers, unlike techniques which operate on the whole tissue slide, where the abnormality could be masked by the large preponderance of cells with normal proteogenomic patterns.

Current methods of cancer diagnosis, and in some cases cancer prognosis using histopathology include trained pathologists examining sample slides from a patient. The pathologists examine patient cells and look for patterns and other markers as identified in one or more SOC trade guidelines, such as guidelines published by the national comprehensive cancer network (NCCN). Pathologists identify type of cells, they are observing in the sample, as well as identifying whether a patient sample contains benign or malignant tumor cells, and in some cases, a grading of the detected cancer cells. The SOC guidelines are typically generated by researchers and health care professionals who through their years of experience observing patient samples have accumulated a knowledge-base of correlations between features in patient sample tissue and cases of other patients in the past and an associated outcome with the observed features or combination of several specific features. In this paradigm, the identification of biomarkers is limited to the guidelines and past experiences of the healthcare professionals. The process of updating the guidelines and the way the pathologists scan, examine and identify biomarkers is therefore a dynamic and at the same time a slow process.

In other words, the current methodologies of biomarker identification can include matching features from a sample space against a limited-scope database of known biomarkers. The described embodiments, on the other hand, can utilize unsupervised artificial intelligence architectures to scan tissue sample image data at a much faster speed and also identify biomarkers predictive of patient outcome that has never been previously identified.

Another challenge with traditional methods of identification of biomarkers and drug target is that diseases, such as cancer can be highly heterogenous and evolving over time. One tumor may include many different molecular subtypes some of which may be biomarkers predictive of patient response. Many techniques look at a small subset of potential molecular subtypes by analyzing a whole tissue slide from a patient. That approach has identified some useful biomarkers, but a wealth of data and information in each patient slide also remains unexamined. As a result, many patients still get baseline treatments, even though they may be good candidates for a different treatment option. Not knowing the relevant biomarkers, the success rate of many treatments is lower than maximum because a large patient population are treated with the same treatment options, without regards to the anticipated response. What is worse, is that in the absence of better alternative, low-success rate treatment options become SOC. Systems and methods that can identify biomarkers predictive of patient response will help to identify patients, who are good candidates for a specific treatment option and deliver targeted and personalized therapies to an individual patient.

Furthermore, patient outcome and responsiveness can be a multimodal problem, where tumor alone or normal disease pathways and mechanisms may not be the only relevant factors. For example, a tumor micro-environment (TME) can play a significant role in patient responsiveness. A drug can be correctly designed based on a disease or tumor, but it might not reach the correct target in the patient if the drug is not designed with the TME of the tumor cells in mind. As an example, a drug might be correctly designed to activate immune system, but in some patients, the tumor might have few infiltrated immune cells, or might have immune suppressor cells nullifying the drug effect. The described embodiments use, the TME of a cell, including stroma, immune cells, blood vessels etc., as well as the tumor cells, when identifying biomarkers, thus enabling the selection of treatment options with higher success potential.

In one sense, the traditional methods of biomarker identification have relied on molecular biologists and pathologists as the initial actors that identified the biomarkers. The results of human-driven identification of biomarkers predictive of patient response were then verified using bioinformatics and statistical analysis. As discussed earlier, the human-driven method of biomarker identification is necessarily limited in the size and number of patient samples capable of being analyzed in laboratory settings, and by the patterns and structures that have previously been identified in research and trade guidelines. The disclosed embodiments, on the other hand, analyze tissue samples in a patient or patient population and identify biomarkers predictive of patient response that may not have been previously known. The results, including the newly identified biomarkers, can be further confirmed by pathologists or biologists in a laboratory setting.

For pharma and oncologists, each stage and sub-type of disease and each potential drug is a unique challenge for biomarker discovery and drug development. The disclosed computer aided systems and methods that correlate disease outcome with tissue morphology are agnostic to the type of cancer and of its treatment. The systems and methods rank morphological features based on known patient outcome to a particular drug to treat a specific disease but do not depend upon the drug mechanism itself. They can therefore be applied to any disease such as cancer that changes morphology and the treatment of interest.

In another embodiment, the method can also be used to identify morphometric features on patient tissues that correlate with a particular molecular change, such as protein loss or gene mutations without the need for a molecular test such as immunohistochemistry (IHC) or gene panel testing. It can rank the morphological features in an unsupervised manner using only the molecular status as label and determine the lead morphology features that can be related to the molecular change.

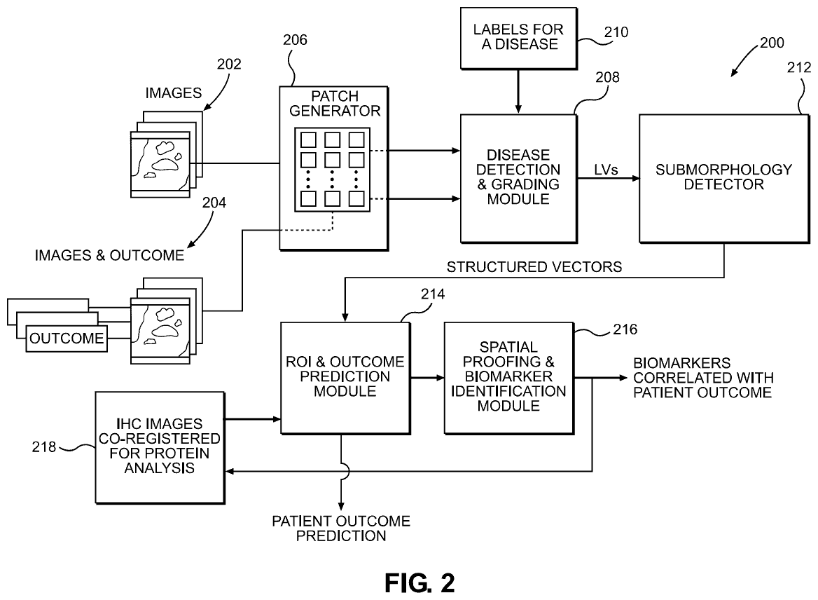

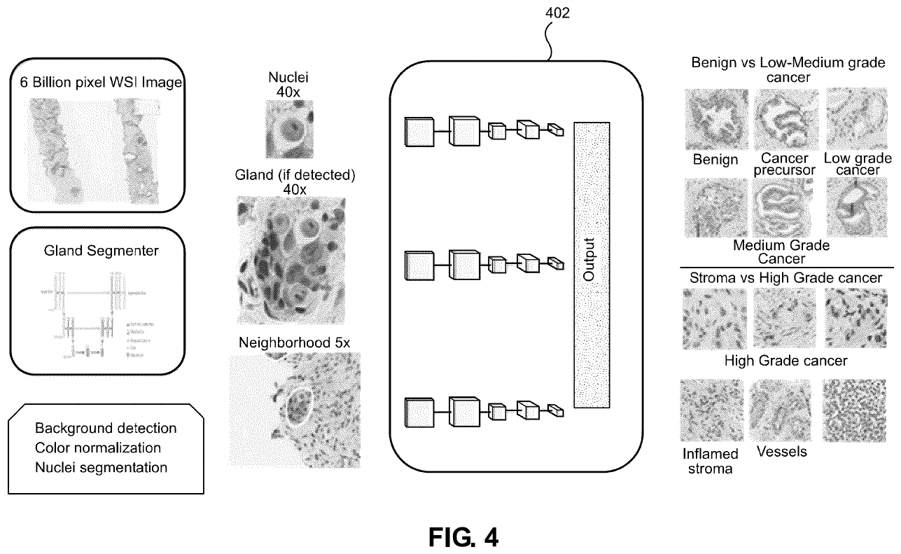

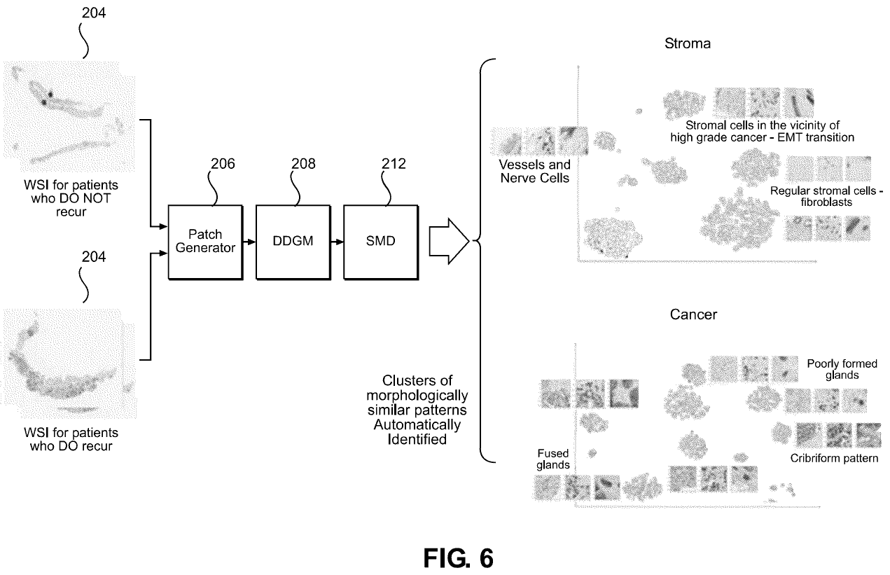

FIG. 2 illustrates a patient outcome prediction system 200 according to an embodiment. Images 202 containing patient data can be input to the system. Images 202 may be the whole slide images (WSI) of patient tissue sample stained with Hematoxylin & Eosin (H&E) at various magnifications, including but not limited to 10×, 20×, 40×, etc. In other embodiments, any image containing patient data, such as X-Ray or IHC images can be input to the system. Some images 204 can include an indication of patient outcome with them. For example, for every image 204, a meta data in the image file or some other form of data-association can indicate whether the patient successfully responded to a treatment or whether the patient did not respond well to a treatment, or other patient outcome data. A patch generator module 206 disclosed here can divide the images into tiles or patches. The sizes of the patches can depend on the implementation and the size of the original images 202 or 204. As an example, the image (shown in FIG. 4 or 6) can be divided up into patches of various sizes, including but not limited to 256 by 256 or 512 by 512 pixels.

Next, a disease detection and grading module (DDGM) 208 transfers the image patches into a vector representation. The DDGM 208 can receive labels 210 for a given disease and using supervised artificial intelligence techniques can determine which label applies to a given patch and augment a vector representation of the patches with applicable labels. These labeled vectors are input to a sub-morphology detector 212, which can use unsupervised learning to determine further morphological sub-patterns within the labels 210. The sub-morphology detector 212 can output structured vectors what include vector representations of image patches labeled with labels 210 and morphological sub-patterns determined by unsupervised learning. The structured vectors outputted from sub-morphology detector 212 are input to a region of interest (ROI) and outcome prediction module 214, which can rank the patches in terms of patient disease outcome and based on whether the determined morphological sub-patterns to which a patch belongs occur in patients with adverse outcomes or patients with good outcome or response treatment. The ranking includes assigning the patches a patch-level score. The patch-level scores can be combined to arrive at a patient-level score indicative of a prediction of a patient's response to a treatment. In one embodiment, a patch-level score can be a number between 0 and 1, where a high score (approximately 1) reflects that the morphological sub-pattern detected for a patch, only shows up in patients with adverse outcome, while a low score (approximately 0) reflects that the morphological sub-pattern only shows up in patients with good outcome.

Patch-level scores can also be used to determine regions of interest on patient's tissue for which further focused laboratory, biochemical or biomarker identification analysis yields information about predicting patient response and/or fine-tuning the artificial intelligence models within the system 200 and/or the ROI and outcome prediction module 214. The regions of interest can capture data on various patient tissue, such as tumor, immune and stromal cells and as a result the ROIs can capture both tumor heterogeneity as well as tumor micro-environment (TME) elements that are prognostic or predictive of the patient outcome.

The ROIs can be input to a spatial profiling and biomarker identification module (SPBI) module 216, where molecular analysis is performed. The molecular analysis is performed on the ROIs to capture differential expression of proteins/RNA in the regions marked as ROI versus regions not marked as ROI. The correlation is done on ROI vs non-ROI of patients with adverse outcome, as well as between patients that have different outcomes to identify the protein/RNA markers that are driving the patient outcome.

In some embodiments, an IHC or immunofluorescence (IF) module 218 can be used. The IHC/IF biomarker slides can be generated for the protein markers identified by SPBI module 216 to capture the spatial distribution in the TME. These biomarker slides can be co-registered with the H&E slides (or other type of input images if used) to determine patch-level biomarker quantification and distribution, as well as other prognostic or predictive data. The combination of biomarker expression (quantity) with morphology data (morphological sub-pattern indication identified by the sub-morphology detector 212) can be used to further improve the accuracy of patient outcome prediction by various modules of system 200, including the ROI and outcome prediction module 214.

In one aspect, the system 200 reduces the complexity of data present in a patient image slide to a data structure. Images 202 and 204 can be WSI or any digitized version of patient tissue, bone or other anatomical regions included but not limited to biopsied tissue, resected samples, circulating blood cells, etc. The images can be divided to a range of 50 to 100,000 patches in some embodiments. These patches can contain different expressions of tumors, benign cells and the microenvironment of the cells. In one sense, the input to the system 200 can include a vast and complex dataset of images containing millions of cells and thousands of patches. The system 200 analyzes this complex dataset and transfers it into structured and usable data for disease and patient response prediction, in one aspect, by determining morphological similarity and determining a more limited dataset of morphological sub-patterns within which these millions of cells and thousands of patches may be classified.

The system 200 performs vectorization on the input image patches and captures morphological similarity between those patches by performing vector operations on the vectors resulting from the patches. In one respect, the tissue space observed in image slides for a given disease (e.g., a tumor type) can be broken down to distinct categories of morphological sub-patterns. For example, within a broader morphological pattern labeled by labels 210. For example, one label 210 might be cells that have morphological patterns of benign cells, while there may be 50 morphological sub-patterns of those benign cells which can further classify those benign cells with more granularity and precision. In routine clinical practice, those morphological sub-patters may be unknown or not labeled to increase the reading efficiency of human pathologists. Nonetheless, those sub-patterns can contain valuable and more targeted information to treat disease or predict patient outcome. The system 200 can determine these sub-patterns within a given label and reduce the complexity of the data.

The input images 202 may be, without any associated patient outcome and for the purpose of training the artificial intelligence networks of the system 200 to identify various morphological sub-patterns. On the other hand, input images 204 may include patient outcome data, so the system 200 can additionally identify whether the detected morphological sub-patterns occur in patients with good or adverse outcomes. In the case of cancer, patients who respond well to a treatment, express the cancer on their tissues in morphologically different ways than the patients who do not respond well to the treatment. Identification of morphological sub-patterns that occur only in patients with good outcome versus those that occur in patients with poor outcome can act as a marker or signature of the category to which a patient might be predicted to belong. Consequently, the detected morphological sub-patterns identify signatures or signals indicative of patient outcome or response, which is used to predict patient outcome or response at an earlier stage of a disease.

In another aspect, the system 200 reduces the complexity of the input data and the overall problem of identifying morphological markers predictive of patient response. For example, at the stage of dividing the input images into patches, hundreds to thousands of patches might exist per patient where thousands of patients might be participating in a treatment program or in a clinical trial. Each image has in the order of gigabytes of data per image. The system 200 reduces the complexity of the input data and the biomarker/morphological identification to, for example, tens of thousands of morphological sub-patterns, where every patient data can be expressed in terms of structured vectors including identification of detected morphological sub-patterns. Vectors can be analyzed between patients with good outcome and patients with poor outcome. Patches with high predictive value and low predictive value can be identified. For example, patches belonging to morphological sub-patters occurring only in patients with adverse outcomes (e.g., failing treatment) can be given a high score. An artificial intelligence model can be trained to rank patches and assign them scores, based on known patient outcomes. The model can learn which patches only occur in patients who fail treatment, which patches only occur in patients who respond well to treatment, and which patches occur in both groups (and are therefore of low predictive value). Accordingly, the model can assign a score to each patch vector and its corresponding vector.

In some embodiments, the disease detection and grading module 208 can be exposed to patient data in two ways. Patient Images 202 do not include patient outcome data. Patient outcome data can be difficult or time consuming to obtain. In some cases, patient outcome data can be available only after following up with a patient 3-5 years after a treatment option is administered. Nonetheless, the artificial intelligence models of the disease detection and grading module 208 can be exposed to input patient image data, without any known patient outcome, for the purpose of training the models to better identify morphological similarity and morphological patterns in patient image data. On the other hand, the disease detection and grading module 208 and the artificial intelligence models therein can be exposed to images 204, where the patient outcome is known, so the models of the system 200 can associate the detected morphological patterns and sub-patterns to a patient outcome and learn how to rank a detected morphological pattern in terms of patient outcome.

In other words, ranking of patches is specific to a particular task, which is specific to a particular clinical question, while detecting morphological similarity (detecting patterns and sub-patterns) can be universal, because a disease (such as cancer) shows up in patient tissue in so many different ways, regardless of the treatment given. Consequently, the models of disease detection and grading module 208 can be improved by exposing them to more patient images, regardless of patient outcome.

In one aspect, the supervised learning models of the system 200 are trained to identify morphological patterns associated with the labels 210 and the unsupervised learning models of the system 200 can identify morphological sub-patterns within the tissue samples labeled by supervised learning models of the system 200. Consequently, the system 200 can identify morphological similarity (morphological patterns and sub-patterns) through unsupervised networks across a population of patients for a given disease. Input images 204 that include patient outcome data can be processed to rank each patch within those images with a score indicative of patient outcome. The patch-level scores can be combined to yield a patient-level score for each patient. The patient-level score indicates a prediction of the patient response to a given treatment.

Additionally, patch-level scores yield ROIs that are candidates for more analysis, both for training the models of the system 200 and for predicting patient outcome or response. Instead of performing molecular analysis of a whole patient image slide, only the regions of an image that contain morphological patterns and sub-patterns that only occur in one group of patients, are analyzed in more detail to accurately yield biomarkers predictive of patient outcome. At the same time, the ROIs identified by the models of the system 200 include the environment and the context of cell tissues to make an improved analysis of those regions, taking into account the heterogeneous nature of cancer. The same tumor cells can look and behave differently across different regions of the tissues, because cancers and diseases are not uniformly mutating or evolving across different sites. Similarly, the microenvironments of the cells also can look different depending on where in the tissue they are from. There may be different types of immune cells, blood vessels and stromal cells that appear in the images 202 and 204 differently depending on the region of the tissue they are from. Consequently, the ranking and scoring of image patches in the system 200 can be at least partially based on the environment and the microenvironment of the cells.

As discussed earlier, the system 200 makes possible analyzing regions of interests in a tissue, as opposed to a whole slide analysis of the tissue. In other words, the system 200 narrows down the field of view to regions that have predictive value. Molecular analysis of those regions can identify the biological mechanisms and pathways that are driving a detected phenotype (morphological pattern or sub-pattern). In other words, the visible regions on patient image slide (such as an H&E image slide) is a manifestation of a tumor, but there are biochemical changes within those cells captured by the image slide that constitutes the basis for the manifestation or morphological patterns or sub-patterns that have appeared in a patient image slide. Identification of those biological processes and pathways through molecular analysis allows for accurate identification of predictive biomarkers as well as developing drugs and treatment options that target those pathways and/or explain why patients respond or do not respond to a given treatment. Many different techniques can be used for further analysis of the ROIs in detail. These can include IHC imaging, IF imaging, genetic profiling and other techniques. These techniques can identify tumor cells, immune cells or other cellular and subcellular components and molecules in the ROIs and mutations or evolutions in those cells. Such identifications can be used for further refinement of the predictive models of the system 200 and/or for better understanding of the disease or patient outcome response to a given treatment. IHCs or IFs can be co-registered on the image slide to obtain a quantification of the expressions of the biomarkers of interest. The quantification can be used for refinement of the predictive models of the system 200 and/or for better understanding of the disease or patient outcome.

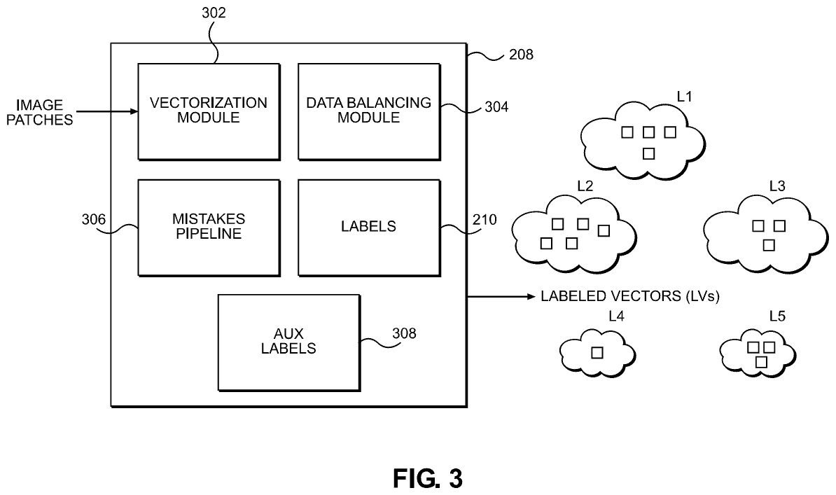

FIG. 3 illustrates the disease detection and grading module (DDGM) 208 and the components therein according to an embodiment. In one aspect, the DDGM 208 transform image patches into vectors. Image patches before vectorization are still a high dimensional data structure. For example, a patch of size 256 by 256 pixels, where each pixel has an RGB value is a data structure of 256×256×3 or a 196,608-value data structure. This high-dimensional data structure can be transformed to a vector of smaller size, using DDGM 208 and sub-morphology detector 212. For example, a patch can be vectorized based on whether the patch is identified to be from a benign tissue and within one of the detected sub-patterns of that benign tissue. In one aspect, the DDGM 208 and sub-morphology detector 212 convert a high-dimensional value patch to a more compact vector. Vectors that have similar mathematical representations can cluster together to identify a morphological sub-pattern.

Pathologists can annotate a set or subset of training images with labels 210. The Labels 210 can be a high-level annotation of morphological patterns that may occur in images 202 and 204. Example labels 210 can include, but not limited to, benign, cancer precursor, low, medium or high-grade cancers, immune cells, stromal cells, etc. The labels 210 can be based on morphological patterns known to pathologists used for cancer detection and grading. A vectorization module 302 can include one or more supervised artificial intelligence networks, including for example, neural networks, deep neural networks, convolutional neural networks (CNNs), and other artificial intelligence networks. The vectorization module 302 accepts as input image patches and using the labels 210 classifies the patches into the categories identified by labels 210. In the example shown, image patches are categorized between 5 labels L1-L5. As described earlier, in some embodiments, the labels 210 can include a high-level disease identification and a high-level grading of the detected disease. The DDGM 208 outputs a labeled vector for each input image patch, that places the patch in a category identified by labels 210.

The labels 210 may be high-level indications of morphological patterns. For example, there may be hundreds of morphological shapes and sub-patterns in which benign cells can appear on an image slide. Labels 210 may be at a high-level or high-abstraction level because labeling all the morphological sub-types that can appear in an image segment can be impractical, burdensome or difficult. Consequently, in regular clinical practice, a pathologist might label and rely on broad labeled categories. The DDGM 208 in combination with the sub-morphology detector 212 can further classify image patches based on the sub-types and sub-morphological categories to which they might belong.

Initially, the sub-patterns are broadly labeled using labels 210 (e.g., benign, cancerous, low, medium or high-grade cancer, immune, stroma) through the supervised learning processes of the DDGM 208. As will be described, the sub-morphology detector 212 uses an unsupervised learning method to extract sub-patterns that may be present in each label 210. In one embodiment, the labels 210 are based on the level of granularity that a pathologist might use to label images or image segments in her regular clinical practice. The artificial intelligence models of the vectorization module 302 can be trained to detect these labels in a set of input patches. In other words, the models of the vectorization module 302 learn morphological patterns and features corresponding to each label 210 and can distinguish and categorize the image patches based on those morphological features and patterns. In some embodiments, the last layer of prediction of the models of the vectorization module 302 can be used to extract a morphological vector corresponding to a patch, which can be used to represent the input data with less complexity, while retaining data relevant to patient outcome or response.

In one aspect, the input image data is heterogeneous and complex. When a pathologist examines an image segment (e.g., an image or image segment of a gland), they look at the nuclei, they look at the gland, and they look at the environment around the gland to make a judgment of whether it is cancerous or not. There may be some cells inside the gland that look cancerous, but the pathologist will still grade the whole gland as benign, if they see some other types of cells, which they know only show up in benign glands. The models of the system 200 and the DDGM 208 perform a similar function. The vectorization module 302 can include auxiliary labels 308 (e.g., nuclei, cytoplasm, gland, neighborhood, etc.). Image patches are also labeled according to auxiliary labels 308. In one embodiment, the models of the vectorization module 302 further classifies the image patches having auxiliary labels 308 at various sizes and resolutions. At the end of a last patch, the models of the vectorization module 302 can for example determine whether a patch has an auxiliary label 308 of a nucleus. The patch is processed through the vectorization module 302 at different resolutions, where it is determined if a label 210 is applicable to the patch, when the patch is viewed at different sizes and resolutions. For example, the vectorization module 302 can determine whether the nucleus is cancerous, benign, whether if the nucleus is in a gland, the gland is cancerous or not, and whether other cells within the neighborhood are cancerous or not. Based on the output of the processing of a patch at multiple resolutions and sizes, the vectorization module 302 can apply or modify a label 210 of the patch accordingly. This is similar to a process that a human pathologist might employ to apply a label 210 (e.g., cancer or not cancer) to an image patch. For example, in clinical practice, if a gland (viewed at high resolution) looks benign, but everything around it (viewed at lower resolution) is cancerous, a pathologist is more likely to conclude that that gland is also cancerous.

In other words, the models of the system 200, including the models of vectorization module 302 can operate on image data in the same way a pathologist might operate on the data (e.g., by labeling the image patches at various resolutions). Consequently, the models learn relevant and effective features, and learn to ignore artifacts. In some embodiments, image patches having been applied an auxiliary label 308 can be processed in the vectorization module 302 at a plurality of resolutions. As an example, an image patch can be viewed by the model and applied a label at three levels of resolution. Fewer or more levels of resolutions are also possible.

Furthermore, the input image data can be highly imbalanced in terms of the features that are relevant to patient outcome or patient response. In the case of cancer, a high-grade cancer may show up in less than 0.01 percent of the tissue. For example, 20-50 cells out of millions of cells on an image slide may be high-grade cancer cells. Those 20-50 high-grade cancer cells can change the treatment decision for the patient. A data balancing module 304 can balance the training data, so the models of the vectorization module 302 can give appropriate weight to morphological features that are highly relevant but may not occur in high frequency or quantity in the image slide. In some embodiments, the data balancing module 304 can use clustering to balance the input training data for the models of the vectorization module 302. The data balancing module 304 can cluster the input data based on morphology through an iterative process. The models of the vectorization module 302 are first trained using the baseline input image data, yielding a first level of accuracy. The output vectors are used to cluster the input image data and pass through the models of the vectorization module 302. Input training data, in subsequent passes, is fed uniformly through the models of the vectorization module 302 in a manner that input data from each cluster can be uniformly sampled throughout the input training data across all pattern subtypes, regardless of how frequently they show up in the tissue.

To further increase the accuracy of DDGM 208, a mistakes pipeline can use patch labels that are determined with less confidence and increase their presence in the input training data, so the models of the vectorization module 302 can better learn the low confidence labels. The vectorization module 302 may use a predetermined label threshold before classifying input data as belonging to a label. For example, one classification threshold of a label can be data having a score of 0.5 or more on a scale of 0 to 1. If data is classified with a score of 0.6. That data is classified with a confidence lower than another data, which is classified with a score of 0.9. Classifications with higher confidence score can increase the accuracy of the models of the vectorization module 302. The mistakes pipeline 306 can identify low confidence classification and sample them as input training data for the next round of training. In another embodiment, the mistakes pipeline can also sample from errors, as well as low-confidence classifications. In some embodiments, the mistakes pipeline is applied after the models of the vectorization module 302 have learned the labels that are easy to learn. In some embodiments, a predetermined percentage of the input training data to the AI models of the vectorization module 302 is used to feed input data from the mistakes pipeline (sampled from those input values that have generated output vectors having error or low confidence level).

FIG. 4 illustrates an example implementation and operation of the vectorization module 302. Artificial intelligence models 402 can be trained to identify different morphological subtypes in H&E slides (or other input images if used). The ground truth for the model 402 can be provided by a group of experienced pathologists, who discuss, agree and then identify known morphology features such as benign glands, cancer precursors, low/medium/high-grade cancers, immune cells, stroma, etc. These labels are based on known morphological patterns that pathologists use for cancer detection and grading.

Additionally, as discussed earlier, the models 402 can be trained based on auxiliary labels 308 to provide more accurate identification and classification for the input data labeled with auxiliary labels 308 based on processing that input data at different sizes and resolutions. For example, for each nucleus that is labeled, three patches are generated (e.g., 64×64 pixels at 40× resolution, 256×256 pixels at 40× resolution and 1024×1024 pixels at 5× resolution). The innermost patch captures the nucleus; the middle patch captures the gland and the outermost patch captures the micro-environment of the nucleus. Three parallel CNNs are run to transform the patch to a 1024-dimensional vector. The 3×1024 vectors are combined and classified to one of the known labels. In other embodiments, fewer or more patches based on a detected nucleus can be generated. Other resolutions and sizes can also be used. In some embodiments, the patches are from a WSI image, which can include a large number of pixels (e.g., in the order of millions or billions). The DDGM 208 can include other modules to improve labeling the input data (by auxiliary labels 308 or by labels 210). For example, a background module, color normalization, gland segmenter or nuclei segmenter can be used to label input image patches.

As described earlier, training data can be highly imbalanced. For example, there can be a very small percentage of high-grade cancer (<0.01%) that can determine or change treatment. To balance the dataset, the vectors generated from the first pass of training can be used to divide the input training data into clusters of morphologically similar patterns. This clustering can be performed, so different sub-patterns of input data are equally represented in the input training data (in iterative passes), even though they may not be present in equal amounts in the training dataset, or in clinical practice.

The mistakes pipeline 306 can further fine-tune the models of the vectorization module 302. Patches that the model has detected with low confidence are identified, based on the absolute value of the difference between the confidence level and a predetermined threshold for a classification label. If the absolute value of the difference does not exceed a confidence threshold, the underlying input data or a sampled subset of the underlying data is fed through the mistakes pipeline 306 to the models 402, as part of the training data. Consequently, the models 402 can give appropriate weight to the low-confidence data. Additional patterns that show up in small quantities and cannot be captured through clustering, can also be fed into the mistakes pipeline 306 to further improve the accuracy of models 402.

In one aspect, the output of the DDGM 208 includes a detection of presence and grading of a disease (via classification in the labels 210) and vector representations for morphological types in that disease. These labeled vectors are fed into a sub-morphology detector (SMD) 212.

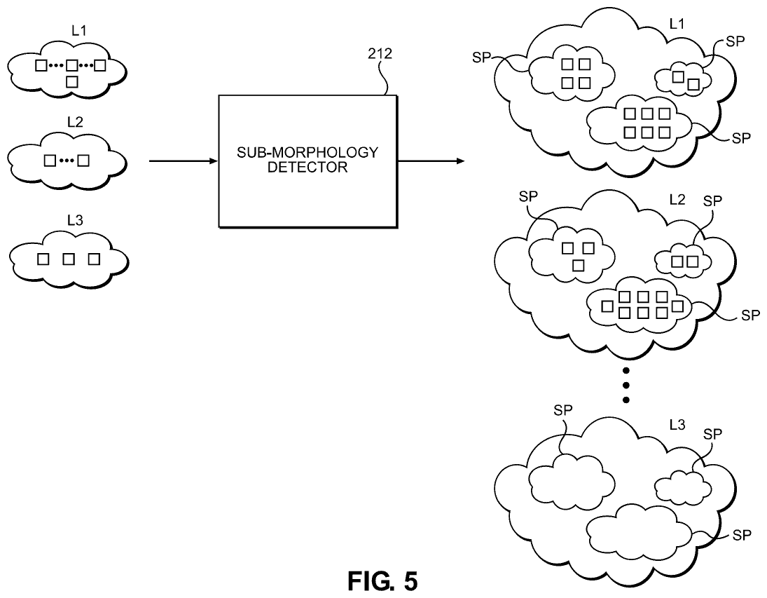

FIG. 5 illustrates a sub-morphology detector (SMD) 212 according to an embodiment. The operations of the SMD 212 will be described in relation to FIG. 5 and previous figures. The SMD 212 receives labeled vectors and detects further morphological sub-patters within each label category, using unsupervised learning algorithms, such as clustering analysis, principal component and other unsupervised machine learning techniques. To provide input to the SMD 212, patient input images 204 that are accompanied by patient outcome data are received. The input images 204 include patient clinical response or outcome data, such as patient response to a drug, response to a treatment option (including surgery, radiation, etc.), whether the patient experienced progression-free survival (PFS), number of months or years of PFS or any other outcome or response criteria that may be of interest to healthcare providers, and disease or treatment researchers.

Each patient slide is divided up to patches using the patch generator 206. As an example, each patient slide may be divided into 50 to 100K patches. Each patient patches and the patient outcome data are fed through the DDGM 208, which generates a labeled vector for each patch. Now, instead of a patch being an image, the patch is represented by a vector. The dimensions of the vector can be chosen according to an embodiment. Examples include 500, 1,000 or 2,000 dimensional vectors. These vectors can inherently capture similarities, which correspond to morphological similarity on a tissue slide. In other words, patterns that are morphologically similar, have mathematically similar vectors. In one embodiment, for example, the cosine function can be used to cluster similar vectors. Vectors that are mathematically similar yield cosine function results that are close in range. Other vector operations can also be used to determine similarity between vectors. In one respect, the SMD 212 converts an image into a structured dataset that can be used to rank and score image patches in terms of patient response or outcome.

FIG. 6 illustrates an example operation of the SMD 212 according to an embodiment. In one aspect, the SMD 212 can extract morphological sub-patterns within a label (applied by DDGM 208) by using unsupervised learning techniques. Input images 204 can include WSI from patients with known clinical outcomes, including those whose disease has recurred and those who have not experienced recurrence. Other potential patient outcomes can include initial response, progression free survival (PFS), overall survival (OS) or other criteria of interest to the clinicians and the researchers. Therefore, the input data can include both patients with positive, as well as adverse outcome. For each patient, the H&E WSIs 204 (or other input images 204 if used) are divided into patches by the patch generator 206. In one embodiment, the size of the patches can be 256×256 pixels at 40× resolution. The patch generator can be configured to generate patches of different sizes depending on the implementation and as input to other modules (e.g., to DDGM when patches of different sizes and resolutions are used to label the input data based on auxiliary labels).

Next, the DDGM 208 can predict a label 210 for each patch. In one embodiment, where three parallel CNNs are used to implement the DDGM 208, the last layer of the vectors is extracted, and used as the vector representation of the patch. As an example, in the three-parallel net architecture, if each vector is of size 1024, the last vector chosen for vector representation of the patch is of size 3×1024 or 3072.

Next, the SMD 212 clusters the vectors to identify morphological sub-patterns within each label. This can generate hundreds of clusters of morphologically similar patterns that do may not have an explicit label but can represent a phenotype. In the example shown, multiple morphological sub-patterns are identified for stroma and cancer labels. The morphological sub-patterns identified by SMD 212 can include regions that in turn include biomarkers, signals or signatures of disease or patient outcome that may have been previously unknown in ordinary clinical practice.

The DDGM 208 and SMD 212 convert unstructured data of gigapixel WSIs (or other input images if used) to a structure of clusters of morphological patterns. This structured representation of morphology enables downstream tasks of ranking these patterns and identifying which patterns are prognostic/predictive of patient outcome.

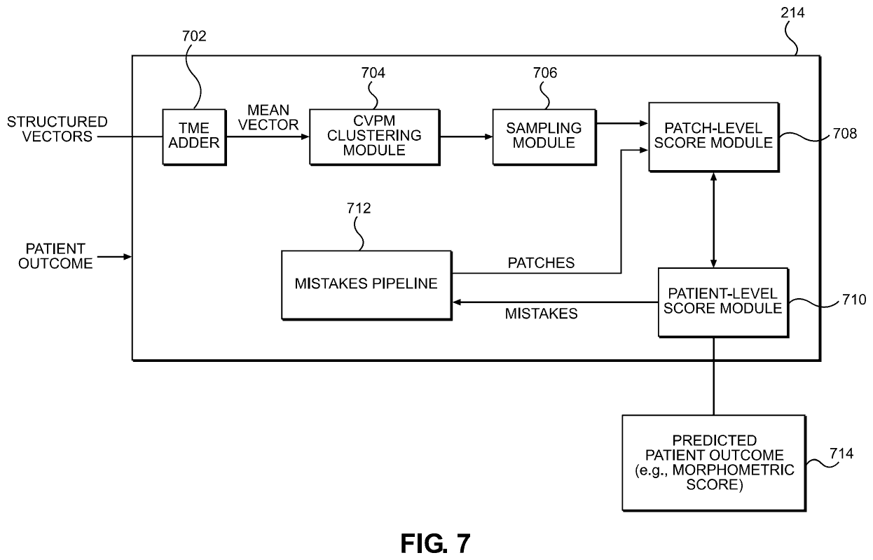

FIG. 7 illustrates the ROI and outcome prediction module (ROPM) 214 and the components therein according to an embodiment. The ROPM 214 receives as input, structured vectors (or structured vectorized image patches) from the conversion of patient image data to structured vectors that identify labels 210 and morphological sub-patterns within each label 210. At patch-level score module 708, an artificial intelligence model can be trained to learn how to rank each patch to arrive at a patch-level score. A patient-level score module 710 can combine patch-level scores to arrive at a patient-level score indicative of a prediction of that patient's response to treatment. In one aspect, the weights of the artificial intelligence model of the patch-level score module 710 are used to arrive at a ranking or score for patches. The ROPM 214 can also receive, as part of training data, patient outcome along with the patient input images, patches and corresponding vectors. The AI model of the patch-level score module 708 fine-tunes its weights when it mis-predicts. In some embodiments, the model minimizes an error function using known patient outcomes as training data. In one respect, The ROPM 214 receive as input structured vectors, which can still be 1000-dimensional vectors and converts them into patch-level scores. In some embodiments, the patch-level score is a number between 0 to 1. The closer the score to 1, the higher likelihood that the underlying morphological sub-pattern contains a signal for adverse outcome. The closer the score to 0, the higher likelihood that the underlying morphological sub-pattern contains a signal for good patient outcome or response to treatment.

The input to the patch-level score module 708 can include a patch and the microenvironment of the patch. In some cases, the patch alone may not capture enough information to accurately score the patch. A TME adder 702 can be configured to obtain a region of predetermined size around a patch (e.g., by using patch generator 206) and vectorize the region (e.g., by using DDGM 208 and/or SMD 212). The region surrounding a patch can be chosen to capture the microenvironment of a patch. The data of the patch and its surrounds (e.g., microenvironment of the patch) can be used as input data to the patch-level score module 708 to generate a score for the patch. As an example, in some embodiments, the microenvironment of a patch can be chosen as a region of 3×3 or 5×5 pixels surrounding a patch. The TME ADDER 702 can combine the vectors from the microenvironment region with the vectors from the patch region. In some embodiments, the combination vector can be a mean vector. Other mathematical techniques combining the microenvironment vectors and the patch vectors are also possible candidates. Combining microenvironment vectors and patch vectors can allow for scoring a patch not only based on any tumor that may be present in the patch, but also the microenvironment of the tumor. The morphological manifestation of a tumor can look different in different regions of tissue based on the cells surrounding the tumor, and the microenvironment of the tumor in general. Consequently, the complexity of the microenvironment of the tumor cells can be captured via combining vectors from the microenvironment region with the patch vectors where the tumor may be present. The combined vectors of patch and microenvironment (CVPM) can be clustered into multiple clusters using a CVPM clustering module 704. As an example, 50 to 100K patches for a patient slide can be converted to 100 to 200 distinct clusters of CVPM. This can substantially reduce the complexity of the input image data and provide further structure for processing of the patient data and arriving at a prediction score.

Clusters of CVPM can include different numbers of corresponding patches because the morphological patterns in each cluster can occur with different frequencies in a WSI image or other patient input image. Nonetheless, frequency of occurrence of a morphological pattern may or may not have relevance to patient outcome. Some infrequently occurring morphological patterns can nevertheless be clinically significant for patient outcome and response. A sampling module 706 can sample input training data from each cluster in a manner that exposes the AI model of the patch level score module 708 to data in a uniform matter, regardless of the volume and frequency of patches in the clusters. In other words, sampling module 706 can be used to input a uniform representation across the morphological subtypes in a patient slide.

The path-level score module 708 can include a deep learning artificial intelligence model that uses its weights to assign a score to each patch. In other words, the AI model of the patch-level score module converts a CVPM to a score (e.g., a number between 0 to 1). A patient-level score module 710 takes a predetermined sampling of the patch-level scores and combines them to arrive at a patient-level score. In one embodiment, a predetermined number of top patch-level scores and a predetermined number of low patch-level scores are combined to arrive at a patient-level score. During training, the patient-level score is compared against known clinical patient output. If the results do not match, the weights of the model associated with the sampled patch-level scores are modified to find a better weight distribution that yields a patient-level score closer to the known clinical patient outcome. In one respect, the combination of all patch-level scores are not used, so the model can remember weights associated with which patch-level scores need to be modified in order to fine-tune the predicted patient-level score. In another respect, the ROPM 214 and the AI model therein learn to predict patient outcome, and in the process, learn to rank patches in a manner that yields an accurate patient outcome prediction. Consequently, patch-level scores can yield regions of interest that likely contain predictive biomarkers, signatures and signals.

In some embodiments, the ROPM 214 includes a mistakes pipeline 712, which is similar in operations to the mistakes pipeline 306 as described above. The mistakes pipeline 712 identifies patches, which the model has not learned well and allocates a percentage of the input data in subsequent passes to those patches, so the model is exposed to and can learn those patches better. The mistakes pipeline 712 can determine which patches correspond to a mistaken prediction, where a mistaken prediction can refer to the output 714 of patch-level score module 708 and patient-level score module 710, at the end of a training pass, predicting an outcome for the patient that does not match the known clinical outcome of that patient. For example, if a patient is non-recurrent, but the output 714 of ROPM 214 is predicting the patient has recurrence. In this mistaken prediction scenario, there are patches that show up on the patient's image slide that the model of the ROPM 214 is allocating high values, where the high values given to those patches can cause the combined patient-level score for that patient image slide to be above a predetermined threshold, and the model is predicting that the patient is recurrent. Conversely, the model of the ROPM 214 may be allocating lower value weights to some patches that should otherwise be scored higher, causing a mistaken prediction of non-recurrent, where the known clinical outcome of the patient is recurrent. The mistakes pipeline 712 can identify patches that are causing mistaken predictions, and feeds those or a sampled subset of them as training input data in successive training passes to the models of ROPM 214. Consequently, the models of the ROPM 214 get exposed more in iterative and successive passes to the patches that the models do not accurately score and learn to score those patches more accurately. In one respect, the mistakes pipeline 712 performs a tuning step, similar to mistakes pipeline 306, where the AI models are first train based on cluster of patches until an initial level of accuracy is reached. After the model is mature and it is still making some mistakes, the training data of the model will be sampled in a way to include a predetermined percentage of mistakes from the mistakes pipeline 712 to train the model for better accuracy regarding those elements that are causing a mistaken output.

The output 714 of ROPM 214 can include patient outcome or response indicator in the form of a morphometric score. In some embodiments, the morphometric score is a number between 0 to 1 that indicates a risk profile of a given patient. The closer the morphometric score of a patient to 1, the higher risk of an adverse outcome for that patient. The output of the patch-level score module 708 also includes scores that indicate risk of correlation between that patch and the patient outcome. Therefore, the patch-level scores can be used to identify regions of interests (ROIs) for further analysis and for finding biomarkers, signatures or signals predictive of patient outcome.

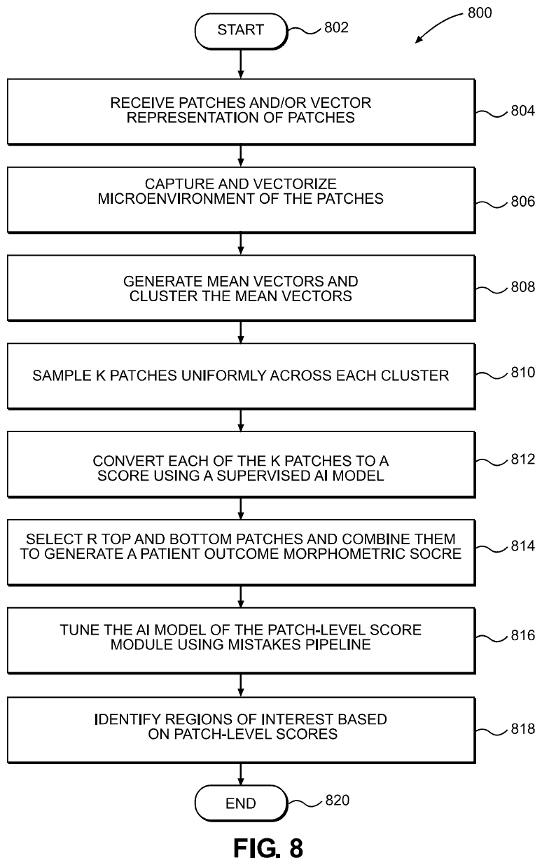

FIG. 8 illustrates a flowchart of a method 800 of the operations of the ROI and outcome prediction module (ROPM) 214 according to an embodiment. The module takes as input patch-level vector representations and generates a rank for each patch based on patient outcome. It can also generate a predictor of patient response or outcome as a morphometric patient outcome score. The method starts at the step 802.

At step 804, for each patient, the patches and corresponding vector representations for the patient image slides are collected. At step 806, to capture the micro-environment of each patch, an N×N region is selected around each patch, and the vectors of those regions are generated. At step 808, the vectors are averaged to generate a mean vector for each region. The mean vectors are clustered per label to generate multiple clusters. As an example, this can convert 50-100 k patches into 100-200 distinct morphological clusters. At step 810, K numbers of patched are sampled uniformly across each cluster to generate a batch of vectors that represent the patient slides.

At step 812, each of the K patches are converted to a patch-level score between 0 and 1, using a supervised deep learning model, which is trained based on patient outcome. A high score (around 1) represents that the patch shows up in patients with adverse outcome, while a low score (around 0) represents good outcome (e.g., the patch appears in patients with non-recurring cancer). The patch-level scores are generated using a set of weights in the deep learning model that are learned by the outcome based on known patient outcome as labels.

At step 814, the top and bottom R patches are selected and combined to generate an outcome morphometric score for the patient. As described above, choosing a limited predetermined set of patches to be responsible for the outcome morphometric score can force the deep learning model to learn the most predictive features and give them the highest or lowest scores. At step 816, the deep learning model is further fine-tuned using a mistakes pipeline. Patches are identified that are causing mistakes in predicting patient outcome. These patches are collected and used to generate training data to expose the deep learning model to learn the mistakes better and assign more accurate weights to them. At step 818, based on patch-level scores, regions of interest (ROIs) are identified on the tissue slide. The method ends at step 820.

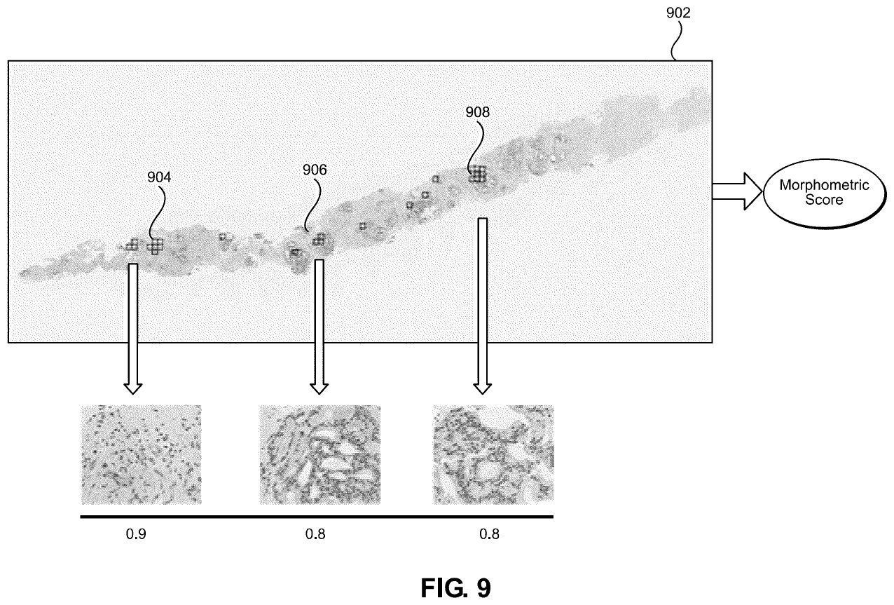

FIG. 9 illustrates a tissue slide 902 that has been processed through the patient outcome prediction system 200. Various clusters of morphologically similar features have been identified. The regions 904, 906 and 908 have been assigned a high patch-level score, indicating those regions are likely strongly correlated with adverse patient outcome (in the form of a morphometric score showing high adverse outcome risk). The regions 904, 906 and 908 are therefore regions of interest (ROIs) that can be further analyzed for determining potential drug or treatment targets and/or biomarkers indicating patient outcome or response. Without identification of ROIs, such as regions 904, 906 or 908, a pathologist would have to analyze the entire tissue slide 902 to determine information related to patient outcome or treatment response. This analysis can typically involve techniques, such as DNA/RNA and protein analysis that would be too burdensome or impractical to perform for a patient. Instead, given the ROIs 904, 906 and 908, a pathologist's effort can be focused and targeted to a much smaller number of cells.

Having identified ROIs, molecular analysis or other techniques can be applied to those regions to determine which gene mutations or cellular processes are causing the patient to exhibit the morphological regions that are indicative of adverse outcome. This platform allows classifying tissue globally based on the protein and mRNA expression in ROI obtained through unsupervised morphological features extraction or focus on any region of interest to discover novel gene expression profiles. Combining gene expression profiles/signature found through protein and RNA analysis with morphological context of ROI (clusters of regions 904, 906 and 908) and non-ROI (other regions or patches which shows low score) in a wide variety of tissue types and their correlation with patient survival or therapy response outcome helps to discover precise biomarker signature for predicting outcome. For example, spatial profiling and biomarker identification techniques can be applied to the ROIs.

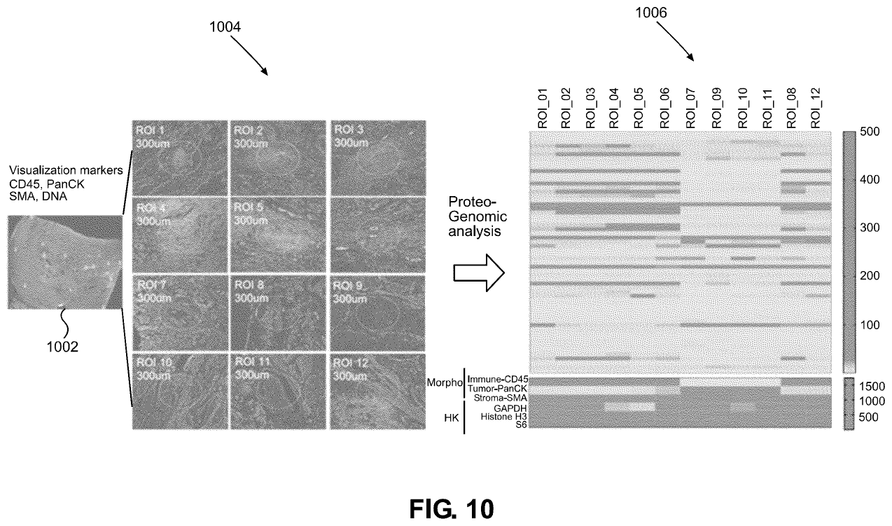

FIG. 10 illustrates an application of proteogenomic technique to regions of interest identified by ROPM 214 to determine proteins or DNA/RNA markers that correlate with patient outcome. The ROIs can be stained with multiple epithelial, immune and stromal markers to identify protein/RNAs that are over- or under-expressed in the ROIs compared to non-ROIs. These markers capture molecular pathways that are driving or at least correlate with the morphological changes in the ROIs and can be predictive of patient response or outcome. Included in the analysis are different types of nonmalignant cells that may be constituents of the tumor microenvironment such as immune and stromal cells. These cells are known to differentiate into subtypes with distinct roles, and this process involves in reprogramming to satisfy their cell autonomous demands and enable interactions with other cell type. Different ROI has same or different proteins/RNAs signature-based pathway enrichment specifically expressed in these specific cell types. In one embodiment, gene set enrichment analysis (GSEA) is performed to compare gene expression between two different subtypes or ROI correlated to patient outcome and to identify different pathways enriched in each subtype/ROI. Significant enrichment of several pathways that distinguished two different cell types or ROI is found, and enrichment of one particular pathway in one or more ROI compared to non-ROI or correlated to patient outcome is found.

In the example of FIG. 10, the tissue slide 1002 is processed through the described embodiments and 12 regions of interest (ROIs) 1004 are identified. Proteo-genomic analysis on the ROIs vs. non-ROIs regions of the slide 1002 is performed as shown in the graph 1006, yielding differential expressions of protein/RNA between ROIs and non-ROIs. The ROIs can be obtained from patches having high patch-level scores near 1 and non-ROI regions can be obtained from patches having low patch-level scores near 0. The differential expressions of protein/RNA can identify biomarkers (e.g., proteins) that are over- or under-expressed in the ROIs vs. non-ROI regions.

Proteogenomic analysis or other biomarker identification techniques allow for detection of markers, including but not limited to immune markers, cancer markers, stromal markers, etc. that may be over- or under-expressed in ROIs compared to the non-ROIs. Without the benefit of ROIs determined by the ROPM 214, molecular markers are searched for at the whole slide level, while the information, related to differential (up-regulation/down-regulation) expression of genes related to specific pathways in ROIs vs non-ROIs and proteins to remodel the tumor microenvironment, is lost or weakened at the whole-slide-level analysis. This suggests that subpopulations of immune cells in the tumor microenvironment have specific features that differ from their behaviors in normal tissues and identify phenotypes that potentially help establish their roles in interacting with other cell types and modulating the tumor microenvironment. On the other hand, the problem with searching the whole slide for biomarkers, as is currently used in some existing techniques, is that any present disease signature is averaged out over the whole tissue, weakening the disease signal by including elements that are not biomarkers, weakening or hindering detection of biomarkers.

When biomarkers (e.g., proteins) predictive of patient response or outcome are identified, it can be beneficial to determine the spatial distribution of those biomarkers across tissue slides. Spatial distribution data can help improve the signature, because there may be elements that are not visible on an H&E slide but may be visible on a biomarker-specific IHC or IF stained image slide that captures spatial distribution of a biomarker. In other words, there may be more relevant or subtle patient outcome or response data that are only visible on biomarker slides. Without the benefit of having identified biomarkers predictive of patient response, it would be burdensome or impractical to develop biomarker slides for all potential biomarkers (e.g., in some cases 20,000 proteins can be potential biomarker candidates. Developing biomarker slides for these many proteins would be burdensome or impractical. Once the set of potential biomarkers is narrowed down, the spatial distribution of those signals can be developed by overlying or co-registering an annotated H&E slide with a biomarker slide, such as IHC or IF, where the annotations include patch-level morphological patterns. From the overlay, tumor cells and the proteins expressed by them are identifiable. The predicted patient outcome or response can be explained biologically as well.

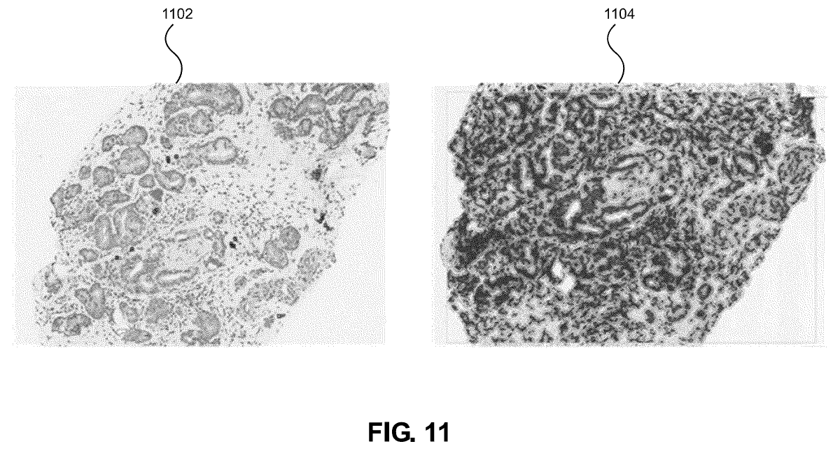

FIG. 11 illustrates an H&E image slide 1102 annotated or co-registered with patch-level morphological patterns, using the described embodiments. Biomarker slides can be developed for the annotated patch-level morphological patterns and for regions that have obtained a high patch-level score, as an example, indicating biomarkers predictive of patient response and/or outcome. The biomarker slides can be developed using a variety of techniques. As an example, the biomarker slide 1104 is generated using IHC imaging of two parallel FFP sections. The biomarker slide 1104 is superimposed with the annotated morphological patterns (or slide 1102) to allow for identification of spatial distribution of the biomarker for which slide 1104 was generated. Generating biomarker slides, such as the biomarker slide 1104 at a whole-tissue level and for every protein candidate can be burdensome or impractical, but given the benefit of the patch-level scores of the described embodiments and the identification of a more limited set of candidate biomarkers, the biomarker slides can be generated and used to gain further insight into the chemical or biological pathways of a disease, treatment or patient response/outcome to disease or treatment. In some embodiments, such insight gained from co-registering the morphologically annotated slide with the biomarker slides can be used to improve the accuracy of the AI models used in the patient outcome prediction system 200.

### Identifying Prostate Cancer Patients at High-Risk of Progression Among Clinically Intermediate Risk Group

The following discussion relates generally to the field of disease detection and treatment identification, and in particular to using artificial intelligence in identifying prostate cancer patients at high-risk of progression among clinically intermediate risk group.

About half of the intermediate group prostate cancer (PCa) patients are stratified into Gleason Grades (GG) 3+4 and 4+3. Several studies show that high-risk patients can still be found in both groups. An AI platform was used to develop a morphometric biomarker by analyzing digitized H&E slide images (WSIs) which predicts early biochemical relapse (BCR) and radiographic progression to metastasis. One hundred twenty five intermediate-risk (n=67, 3+4; n=58, 4+3) samples were collected from the Icahn School of Medicine at Mount Sinai (ISSMS) to form a held-out test set. A series of deep learning models trained using data from ISMMS, the University of Wisconsin-Madison, and TCGA generated a high dimensional vector for each WSI to provide a numerical representation of observed morphologies, which are then converted into a single score to predict BCR within 36 months and high risk of metastasis (MET). Area-under-the-receiver operating characteristic (AROC) was used to measure the accuracy of BCR, and the concordance index (CI) was used to measure the performance of MET. The high and low-risk groups' hazard ratios (HR) for patients within grades 3+4 and 4+3 show that our model can further stratify GG. Our method was significantly better at predicting BCR (AROC: 0.801) and ranking MET (CI: 0.764) relative to standard clinical metrics, GG, pathologic staging, and genomic tools such as DECIPHER. We further sub-stratified the patients into GG 3+4 and 4+3 and identified high-risk patients within each GG. Patients in GG 3+4 with high PathomIQ scores had a significantly higher risk of BCR (HR 3.3; 95% CI 1.44-7.56; p<0.005) compared to the low PathomIQ score patients. A similar trend was seen in the GG 4+3 group (HR 3.0; 95% CI 1.32-6.83; p<0.01). Our histopathology-based prognostic biomarker significantly improves over standard clinical markers in stratifying patients with intermediate-risk PCa for BCR and MET. Our scoring method may strongly impact the management of intermediate-risk PCa patients and clinical trial patient selection for the successful development of new therapies for early-stage PCa.

### Example Data Sources

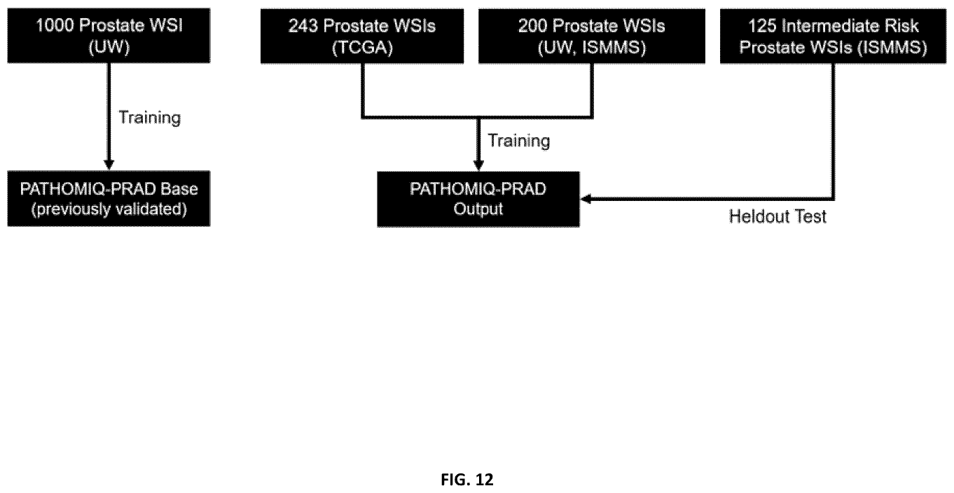

FIG. 12 illustrates a diagram of data usage in some embodiments. Two types of data were collected: (1) WSIs without associated outcome data for the training and development of various AI modules which compose the base of PATHOMIQ-PRAD, designed to accurately identify hundreds of distinct high and low-level morphological features; (2) WSIs with associated outcome and clinical data for the training and validation of the final output of PATHOMIQ-PRAD, a single score indicating risk of biochemical recurrence. This protocol is visualized in FIG. 1. All data were sourced in a deidentified manner as approved by the respective institutional review boards (IRBs). FIG. 12 describes that 1000 outcome independent slides were used to trained the base of PATHOMIQ-PRAD. 243 slides from publicly available data (TCGA) and 200 slides from University of Wisconsin-Madison (UW) and the Icahn School of Medicine at Mount Sinai (ISMMS) were used to train the final outcome model. 125 slides from ISMMS were used as a held out test set.

PATHOMIQ-PRAD Base. A total of 1000 radical prostatectomy (RP) WSIs from 589 patients were collected from the University of Wisconsin-Madison (UW) and 243 from the publicly available Cancer Genome Atlas (TCGA). Slides from UW were scanned at 40× magnification using a high-capacity scanner (Aperio AT2 DX; Leica Biosystems). The training and validation details of models which make up the base of PATHOMIQ-PRAD using these slides have previously been published.

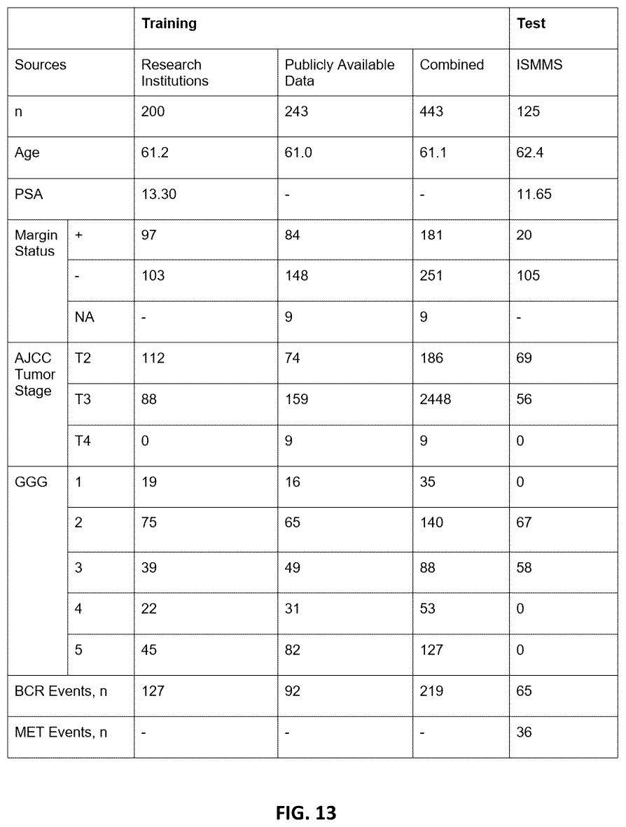

PATHOMIQ-PRAD Output. A total of 325 RP and biopsy WSIs were collected from the Icahn School of Medicine at Mount Sinai (ISMMS) and UW. TCGA slides were also used in this mode. Slides from ISMMS were scanned using Aperio CS2 (Leica Biosystems, Inc.) at 40× magnification. Each WSI was associated with a single patient and included an accompanying time-to-biochemical relapse (BCR), and standard clinical information such as Gleason grade, margin status of the patients post RP, pathologic staging, etc. WSIs from ISMMS also included time-to-metastasis (MET) and subset included DECIPHER scores, a genomic risk profiling tool. The Table in FIG. 13 summarizes the data. The table lists training and test data of a final BCR prediction model. Distribution of Gleason grade group (GGG), biochemical relapse (BCR), and metastasis (MET) from both publicly available TCGA and individual cancer centers.

### Patient Selection

The output of PATHOMIQ-PRAD was trained using data sourced from research institutions (ISMMS and UW) and publicly available data (TCGA). Patients were included in training if they had a BCR within 36 months or no BCR after 36 months. BCR was defined by PSA rising by two consecutive tests post-RP relative to the first PSA level post-RP. Patients with BCR after 36 months were excluded. No patient received perioperative treatment with androgen deprivation therapy or adjuvant radiotherapy, including patients with positive margin.

To most effectively test the predictive power of PATHOMIQ-PRAD in identifying high risk patients, we curated a held-out test set comprised only of patients with assigned intermediate-risk Gleason scores in order to measure its prognostic and predictive power when looking at the difficult to assess cases. This held-out test set from ISMMS had 125 patients with Gleason score 7 (n=67, 3+4; n=58, 4+3).

### Architecture

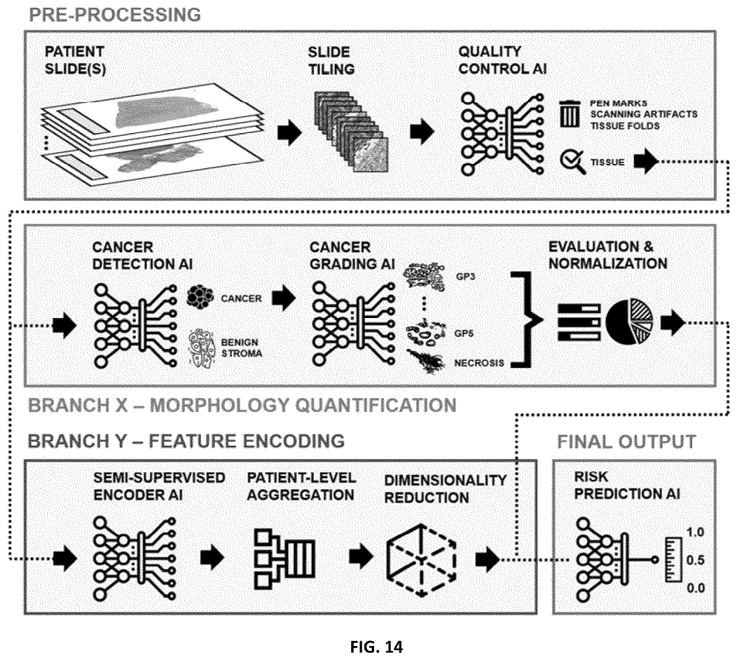

Referring now to FIG. 14, the figure describes an embodiment of processing patient slide data and determining a final output of risk prediction. The patient slide data is tiled and processed though one or more trained machine learning models.

A portion of the system (also referred to as PATHOMIQ-PRAD) comprises multiple stages and multiple AI-modules, each responsible for a different task Branch X is responsible for tile-level classification of tissue and quantification of morphologies; Branch Y is responsible for high level feature encoding of slides. A final model aggregates information from both branches to output a single score which represents risk of biochemical recurrence. All models were implemented in Python 3.9 using the deep learning framework Pytorch, and the models were trained on an Amazon Web Service g3.8×large instance, with 4 GPUs, 64 vCPUs, 16 GB GPU RAM.

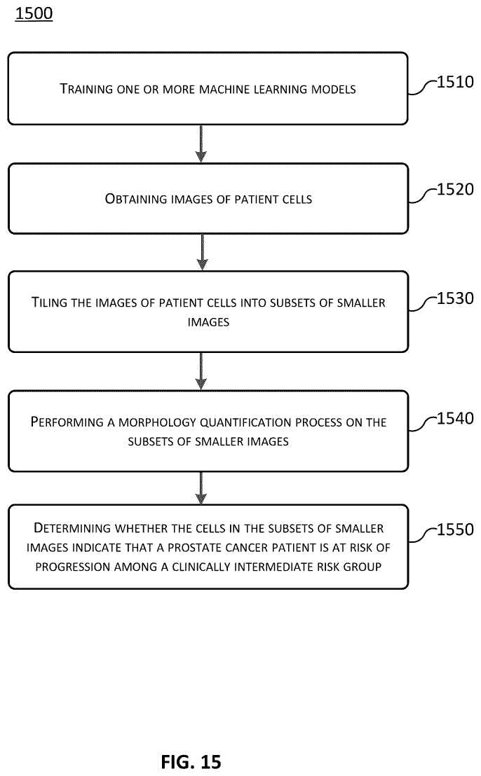

FIG. 15 illustrates a flowchart of an embodiment of a method 1500 performed by the system. The method 1500 performed the general steps as described in the flow chart. Further detailed process in describe below. In step 1500, one or more machine learning models are trained using images of patient tissue cells. In step 1520, images of patient tissue cells may be obtained and evaluated by the system. In step 1530, the obtained images are tiled into subsets of small images. In step 1540, a process of morphology quantification is performed by on the subset of smaller images. For example, the system may input a portion of the images into the trained machine learning model which determines whether cells depicted in the images are likely cancerous cells, and classifies the cells with a grade of GP3, GP5 and/or necrotic cells. In step 1550, the system identifies whether the cells in the subset indicates whether a prostate cancer patient is at risk of progression among a clinically intermediate risk group.

Pre-processing. While the process requires at least one digitized biopsy or RP slide, multiple slides may be submitted for one patient. If submitted slides were not originally scanned at 40× magnification, the slide is upscaled to 40×. First, all digitized tissue across one or more slides is divided into 256×256 tiles and filtered using a quality control AI module which removes tiles with pen marks, scanning artifacts, tissue folds, and other degrading characteristics.

Branch X. Afterwards, each tile is fed through a series of convolutional neural network (CNN) deep learning models in order to classify them for morphological quantification. The cancer detection module classifies tiles between cancer, benign, and stroma. The cancer grading module takes any tile previously classified as cancer and predicts them into various standard prostate-specific morphological patterns. Using these classifications, slide level statistics are generated and normalized.

Branch Y. Each tile is encoded into a high-dimensional feature representation using a previously trained CNN encoder. All patient level vectors are averaged into a single vector and then it is reduced in dimensionality using principal component analysis.

Final Output. In the last step, information from Branches X and Y are combined as covariates in a final survival model which is trained to output a score between 0 and 1 which indicates risk of biochemical recurrence, with 1 being the highest likelihood.

### Nomogram

Nomograms are useful ways to combine different clinical parameters and tests into a unified score. We compared against three of the most popular ones in the prostate setting. Cancer of the Prostate Risk Assessment score (CAPRA-S) was calculated using the pre-surgical PSA level, pathologic grade, Gleason score, positive surgical margin, and histologic markers including extracapsular extension, seminal vesicle invasion, and regional lymph node invasion. Partin was calculated using clinical stage, gleason score on diagnostic biopsy, and pre-surgical PSA. Finally, Kattan was calculated using age, pre-surgical PSA, gleason score on diagnostic biopsy, and pathologic grade.

### Model Evaluation

Area-under-the-receiver operating characteristic (AROC) was used to measure the overall performance of each method's ability to predict BCR within 36 months by evaluating all pairs of sensitivities and specificities using a sweeping threshold. An AROC of 0.5 indicates random performance. Concordance index (CI) was used to measure the performance of each method's ability to rank patients by risk of metastasis in concordance with their actual time-to-metastasis, including censored and uncensored outcomes. Similar to AROC, a CI of 0.5 indicates random performance.

A univariate analysis for each method was conducted using Cox-proportional hazard modeling. Hazard ratios with confidence intervals and statistical significance are provided for each independent covariate for both BCR and MET. The genomic test score was divided into low, intermediate, and high categories using its respective thresholds of 0.45 and 0.6. CAPRA-S was divided into low and high categories based on its point system. Because Kattan and Partin nomograms use continuous scoring systems without designated categorical risk groups, a median threshold was used to define low and high risk categories for the sake of comparing hazard ratio with the other methods.

To measure our model's predictive value in identifying high risk patients within samples assigned intermediate-risk Gleason scores, we stratified patients into high and low risk groups based on our method's output score and using a clinical cut off determined using the median PATHOMIQ_PRAD score on training data. Using this classification, we use Kaplan-Meier modeling to measure and visualize how confidently these groups are stratified from each other. Unlike AROC which uses a sweeping threshold to measure overall accuracy, this single threshold analysis is essential for real-world clinical decision making. The log rank test was used to measure the statistical significance of stratification (p<0.05) between groups.

### Results

All results are measured on the intermediate-risk patients (n=67, GG 3+4; n=58, GG 4+3) held-out test set described herein.

### Predictive and Prognostic Performance

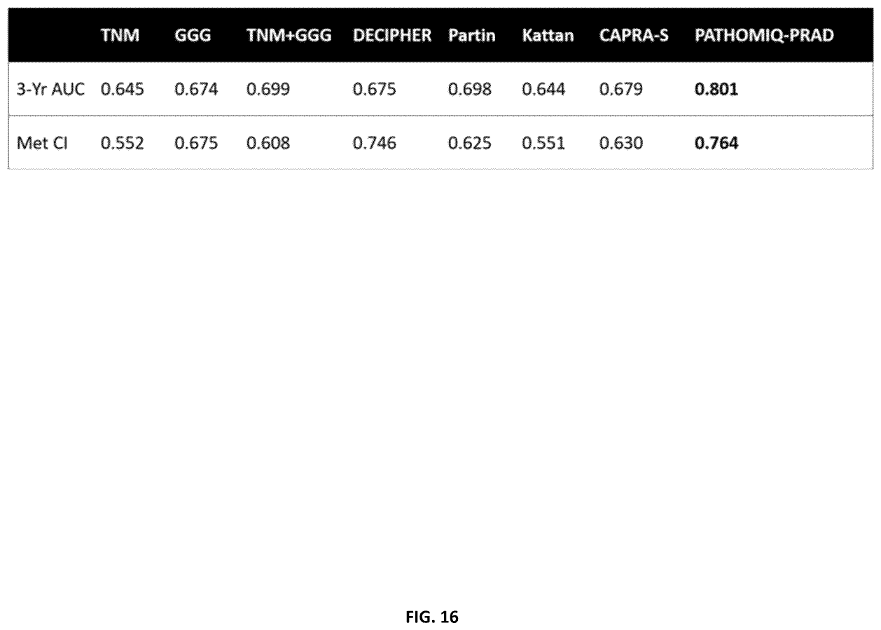

Referring to FIG. 16, a table summarizes the overall performance of PATHOMIQ-PRAD's ability to predict BCR (AUC: 0.801) and metastasis (MET) (CI: 0.763) when compared to other clinical methods across the entire held-out intermediate-risk test set. Not every patient in the complete test set had a genomic score. Therefore, relative performances were calculated on the subset with available genomic score for fair comparison. On the subset with a genomic score, our method (BCR AUR: 0.816, MET CI: 0.808) outperformed Decipher (BCR AUR: 0.675, MET CI: 0.746). The table describes the performance of predicting biochemical recurrence within 3 years is measured by area under the receiver operating index (3-Yr AUC) and performance of ranking time to metastasis is measured by concordance index (Met CI).

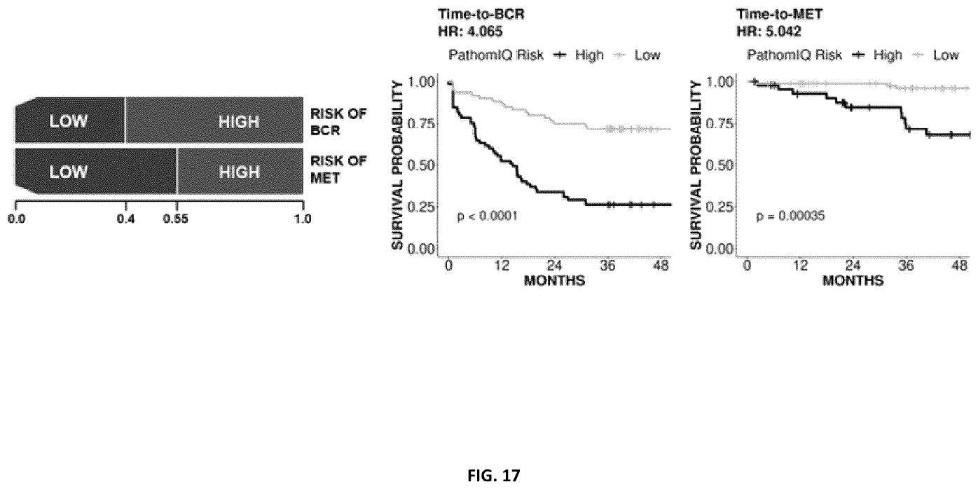

In order to determine specific cutoffs for time-to-BCR and time-to-MET, scores produced on the training data were used to maximize patient stratification in each time to event category. BCR was best stratified using 0.4 and MET was best stratified using 0.55. These thresholds were then used to stratify the held-out test data. FIG. 17 highlights the test data as stratified by each respective cutoff using Kaplan Meier curves. The p-value provided corresponds to the log-rank test which measures statistical significance. BCR was stratified with an HR of 4.065 (95% CI: 2.33, 7.10, p<0.0001) and MET was stratified with an HR of 5.042 (695 CI: 1.88, 13.51, p=0.00035).

### Univariate Analysis

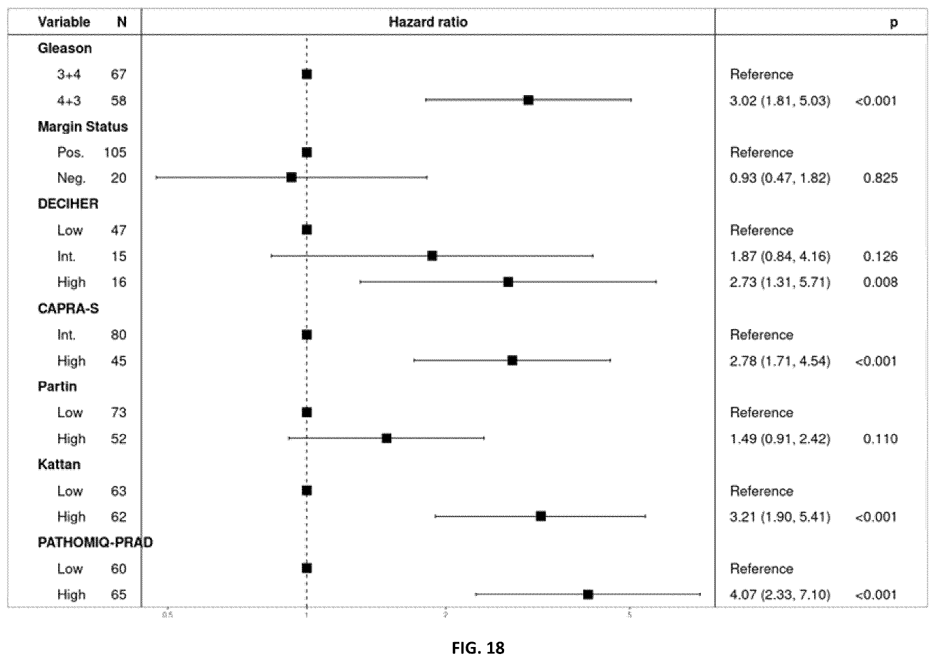

FIG. 18 illustrates a chart describing hazard rations (HRs). The HRs have a 95% confidence interval for each method of stratification on the test set. The HRs were calculated to observe the categorical risk profiles for each method. As a baseline, Gleason 7 can be stratified into 3+4 and 4+3. The other methods were classified into categories based on parameters outlined in the previous section. PATHOMIQ-PRAD had a HR of 4.07 (95% CI: 2.33, 7.10). In comparison, Gleason 7 is stratified into 3+4 and 4+3 with an HR of 3.02 (95% CI: 1.81, 5.05).

### Gleason Stratification

FIG. 18 illustrates multiple Kaplan-Meier plots highlighting stratification within Gleason 3+4 (top) and 4+3 (bottom). To measure each method's ability to further stratify patients over Gleason Grading, we assessed each model's capacity to stratify within each Gleason 7 subgroup. Using our method, 34% of patients were identified as high risk within GG 3+4 (GGG2; HR=3.303, 95% CI 1.44-7.56; p<0.005) and 87% of patients were identified as high risk within GG 4+3 (GGG3; HR 3.0; 95% CI 1.32-6.83; p<0.01). FIG. 5 shows a visualization of Kaplan-Meier plots to highlight the stratification between both groups using our method vs. others. Our method was able to stratify patients and identify high risk patients with statistical significance within both GG 7 subgroups. All nomograms failed to substratify GGG3 patients with statistical significance. Even for the GGG2 subgroup, our method shows a difference of >12 months higher median survival time between high risk and low risk patients as compared to the stratification made by each of the nomograms.

### Summary Discussion

Our method was significantly better at predicting BCR within 36 months after RP and ranking MET relative to GG, TNM, and CAPRA-S. These results demonstrate that our AI-based deep learning method is capable of further dissecting the morphological characteristics of PCa tissue H&E stained images that are not easily discernible by human eyes and thus, never been included in the GG system.

Additionally, our assay is based on digital images of H&E stained slides that does not require shipping of slides as required by all genomic analysis based assays. Moreover, our assay is non-destructive and the tissue can be subjected to other proteo-genomic analysis subsequent to our scoring, if desired.

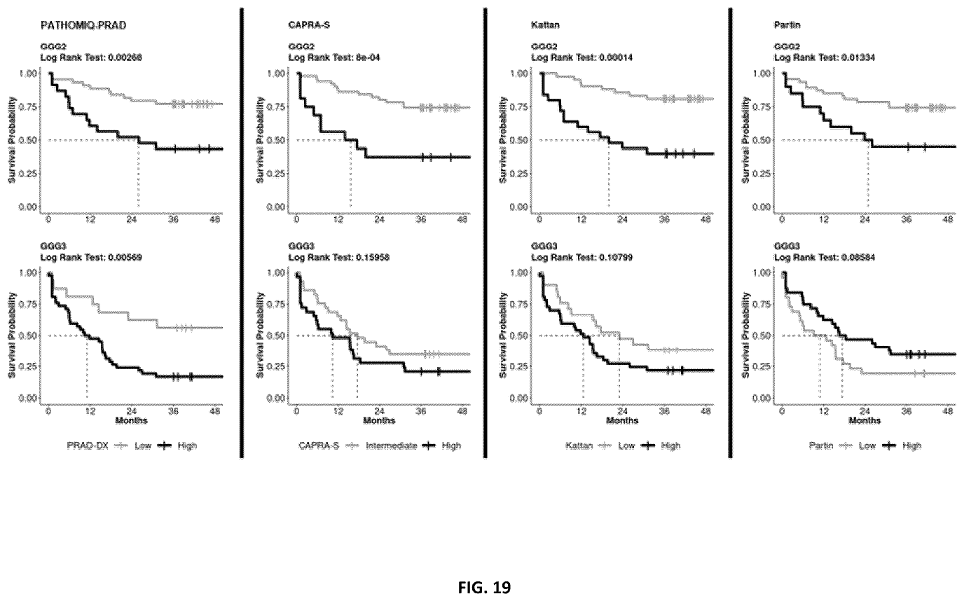

The results illustrated in FIG. 19 clearly demonstrate that our PATHOMIQ-PRAD test can substratify GGG2 (Gleason 3+4) and GGG3 (Gleason 4+3) patients with high statistical significance. It is also more efficient than other methods currently used in the clinic for sub-stratification, particularly for the intermediate-risk category patients. While CAPRA-S cannot substratify GGG3 patients at all, CAPRA-S has some efficacy in substratifying GGG2 patients. However, CAPRA-S stratified GGG2 subgroup has >12 months less overall median survival time than has PATHOMIQ-PRAD stratified GGG2 subgroup.

Thus, PATHOMIQ-PRAD performed on both biopsy and prostatectomy tissue images is a much superior test in predicting both BCR and MET in intermediate-risk PCa patients as compared to other tests generally performed in the clinic today. Some of these proteogenomic tests require physical shipping of tissue samples that are destroyed during the testing process. Use of this this test on all PCa patient tissue images has demonstrated high accuracy of BCR prediction for all PCa patients. That has now been extended to prediction of both BCR and MET with similar accuracy for intermediate-risk patients.

Thus, system and methods described herein will be very helpful in assisting both the urology surgeons and PCa medical oncologists to address the following unmet need in PCa management decision for intermediate-risk patients-(i) deciding on who needs RP and who can be kept in watchful waiting, (ii) deciding on who needs aggressive therapy such as ADT and/or anti-androgen treatment post-RP.

Our systems and methods described herein provide a novel AI-powered PCa prognostic test for a larger cohort of intermediate risk patients undergoing RP that can identify patients who will likely be benefited by aggressive treatment such as ADT or anti-androgen therapy post-surgery to delay or block PCa progression.

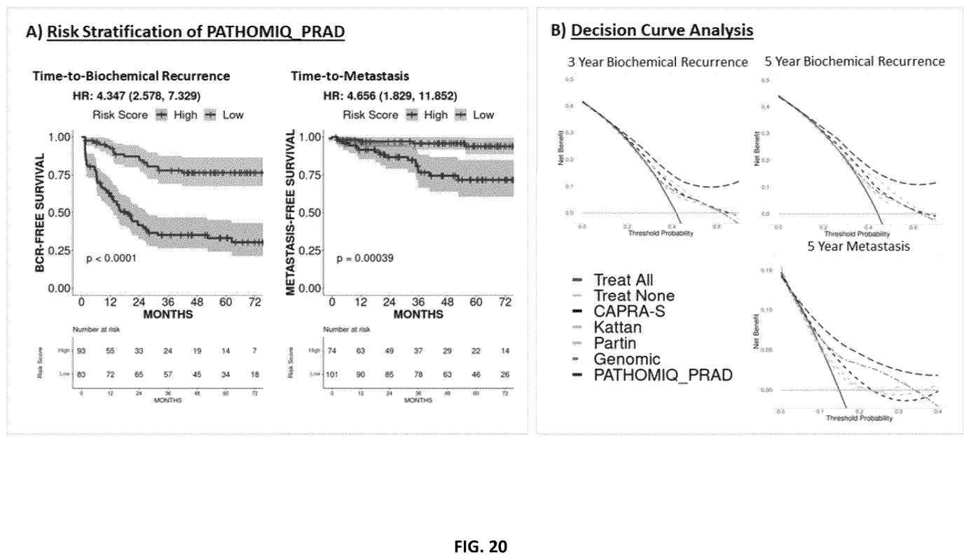

FIG. 20 illustrates the tables illustrating risk stratification and decision curve analysis. FIG. 20, table (A) is a Kaplan-Meier figure that illustrates the relationship between patient outcome in terms of biochemical recurrence and the occurrence of metastatic disease. Patients are categorized into two groups based on their outcome: those with a positive outcome (represented by the top graph) and those with an unfavorable outcome (represented by the bottom graph). This categorization is done using the PATHOMIQ_PRAD system. The hazard ratio (HR) represents the ratio of favorable to unfavorable risk groups, together with their corresponding 95% confidence intervals. FIG. 20, table (B) illustrates a Decision Curve Analysis (DCA) was conducted to examine the effectiveness of PATHOMIQ PRAD, Genomic Score, CAPRA-S, Partin, and Kattan's net benefit in aiding clinical decision-making for the 5-year likelihood of biochemical recurrence (BCR) and 5-year likelihood of metastasis (MET). Additionally, a 3-year probability of biochemical recurrence was included to address a more immediate clinical need. This analysis was performed on a subset of patients (n=129) with a genomic score for fair comparison. The threshold probability indicates the level of risk necessary for a clinician to make a treatment decision post-surgery, including adjuvant therapies. For example, a threshold probability of 30% applies to patients who would be prescribed combination treatment if their risk of disease progression was 30% or higher. DCA for both 3-year and 5-year BCR and 5-year MET confirmed that PATHOMIQ PRAD offers superior net benefit compared to existing nomograms, CAPRA-S, and genomic scores across a range of clinically relevant probabilities.

Additional Discussion of methodologies followed:

### Data Sources

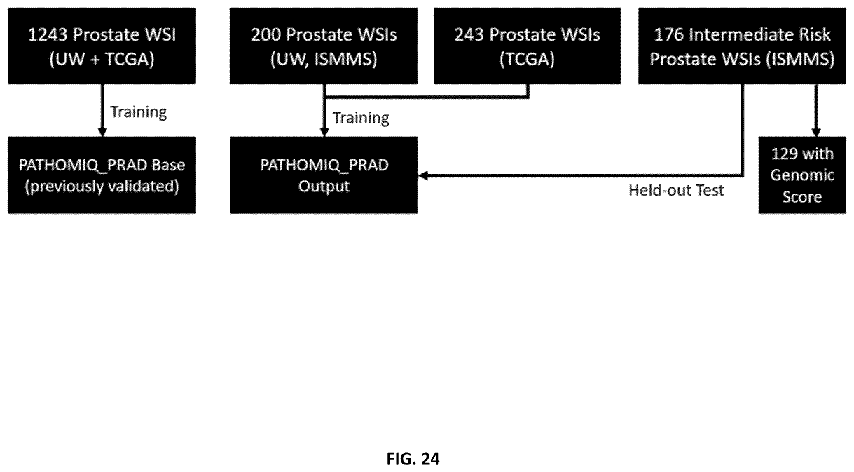

Two types of data were collected: (1) Hematoxylin and Eosin (HE)-stained whole-slide images (WSIs) without associated outcome data for training and development of various artificial intelligence (AI) modules that comprise the PATHOMIQ_PRAD base, designed to accurately identify many hundreds of distinct high- and low-level morphological features; and (2) WSIs with associated outcome and clinical data for training and validation of the final PATHOMIQ_PRAD output, which is a single score indicating the risk of biochemical recurrence (BCR) and metastasis. The workflow is illustrated in FIG. 24. All data was sourced in a deidentified manner as approved by the relevant institutional review boards. Workflow for Prostate Cancer WSI Analysis and Validation Using PATHOMIQ_PRAD. The base was trained with 1000 outcome-independent slides from the University of Wisconsin-Madison (UW) and 243 slides from publicly available data (The Cancer Genome Atlas; TCGA). 200 slides from UW and the Icahn School of Medicine at Mount Sinai (ISMMS) were used to train the final PATHOMIQ PRAD output model. A further 176 slides from ISMMS were used as a blinded test set. Of these, 129 had a corresponding genomic score.

### PATHOMIQ_PRAD Base

A total of 1000 radical prostatectomy (RP) WSIs from 589 patients were collected from the University of Wisconsin-Madison (UW), as well as 243 from the publicly available data in The Cancer Genome Atlas (TCGA). Slides from UW were scanned at 40× magnification using a high-capacity scanner (Aperio AT2 DX; Leica Biosystems). The training and validation details for models constituting the base for PATHOMIQ_PRAD using these slides have previously been published.

### PATHOMIQ_PRAD Output

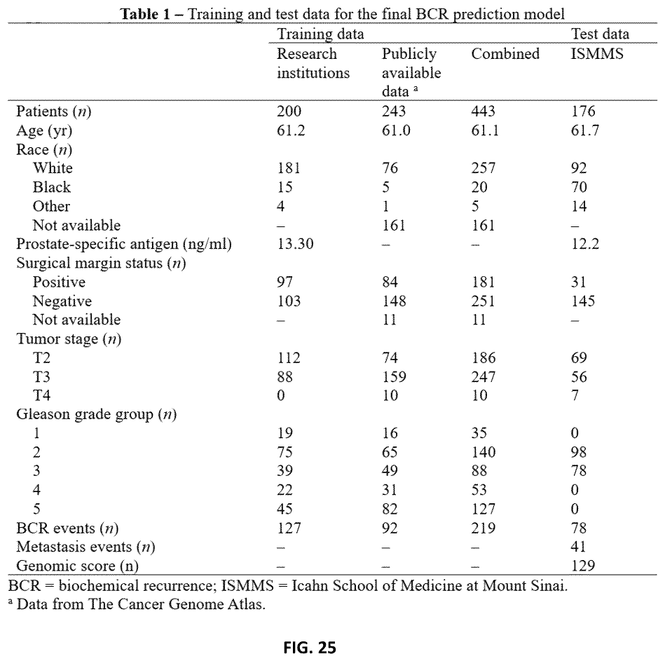

A total of 376 RP and biopsy WSIs were collected from the Icahn School of Medicine at Mount Sinai (ISMMS) and UW. 243 TCGA slides were also used in this mode. Of this data, 176 WSIs from ISMMS were set aside as a blinded test set. Slides from ISMMS were scanned NanoZoomer S210 Digital slide scanner (Hamamatsu USA) at 40× magnification. Each WSI was associated with a single patient and included the time to BCR and standard clinical information such as decision-making nomograms. WSIs from ISMMS also included time to metastasis (except one case) and subset included DECIPHER scores, a genomic risk profiling tool. Table 1 of FIG. 25 summarizes the data.

### Patient Selection

Patients were included in the training set regardless of whether or not they had a BCR event after RP. BCR was defined as rising prostate-specific antigen (PSA) on two consecutive tests after RP relative to the first PSA level after RP. No patient received perioperative treatment with androgen deprivation therapy (ADT) or adjuvant radiotherapy, including patients with a positive surgical margin.

As PATHOMIQ_PRAD was the most effective test in identifying patients with high-risk disease, we curated a held-out test set comprising only patients assigned an intermediate-risk Gleason score to measure its prognostic and predictive power for looking at cases that are difficult to assess. The blinded test set from ISMMS comprised 176 patients with Gleason score 7 (98 with Gleason 3+4 and 78 with Gleason 4+3).

### Architecture

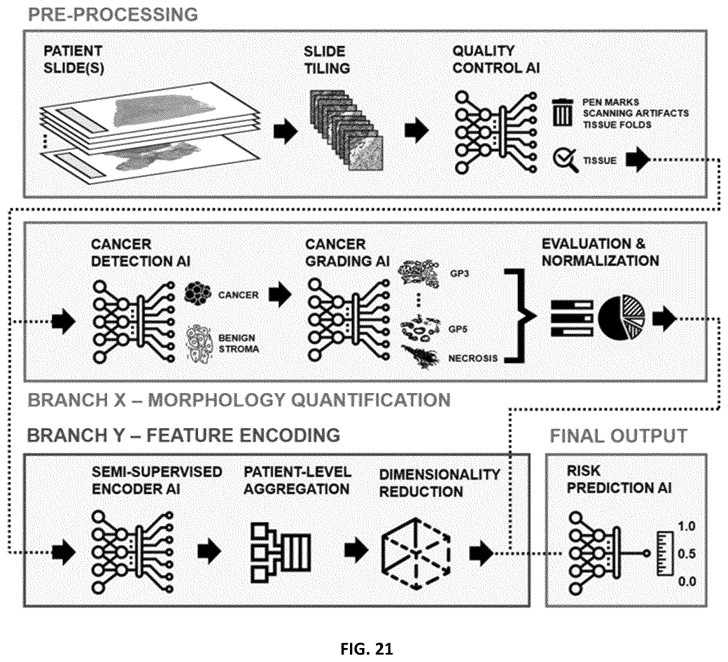

PATHOMIQ_PRAD consists of multiple stages and multiple AI-modules, each responsible for a different task (as illustrated in FIG. 21). FIG. 21 is a schematic overview of the PATHOMIQ_PRAD pipeline. Slides for each patient are tiled and filtered for artifacts using an AI-based preprocessing method. Each tile is then predicted using branch X to quantify tissue and branch Y to extract rich high-level features. Information from the two branches is combined to make a final prediction.

Branch X is responsible for tile-level classification of tissue and quantification of morphologies. Branch Y is responsible for high-level feature encoding of slides. A final model aggregates information from both branches to output a single score representing the risk of BCR. All models were implemented in Python 3.9 using the Pytorch deep learning framework and were trained on an Amazon Web Service g3.8×large instance with four GPUs, 64 vCPUs, and 16 GB of GPU RAM.

### Preprocessing

While the algorithm requires at least one digitized biopsy or RP slide, multiple slides may be submitted for an individual patient. If a submitted slide was not originally scanned at 40× magnification, the slide is upscaled to 40×. First, all digitized tissue across one or more slides is divided into 256×256 tiles and filtered using a quality control AI module that removes tiles with pen marks, scanning artifacts, tissue folds, and other degrading characteristics.

### Branch X

After preprocessing, each tile is fed through a series of convolutional neural network (CNN) deep learning models to classify them for morphological quantification. The cancer detection module classifies tiles between cancer, benign, and stroma. The cancer grading module takes any tile previously classified as cancer and predicts them into various standard prostate-specific morphological patterns. Using these classifications, slide level statistics are generated and normalized.

### Branch Y

Each tile is encoded into a high-dimensional feature representation using a previously trained CNN encoder. All patient level vectors are averaged into a single vector and then it is reduced in dimensionality using principal component analysis.

### Final Output

In the last step, data from branches X and Y are combined as covariates in a final survival model that is trained to output a score between 0 and 1 reflecting the risk of BCR, where 1 denotes the highest likelihood.

### Model Evaluation

To measure the predictive performance of PATHOMIQ_PRAD in identifying high-risk PCa among samples assigned intermediate-risk Gleason scores, we stratified patients into high and low risk groups on the basis of the output score and clinical cutoffs of 0.45 for BCR and 0.55 for metastasis, as used in previous validation studies. We used the Kaplan-Meier method to measure and visualize the stratification of these groups for the complete test set. The log-rank test was used to measure the statistical significance of the stratification (p<0.05) between groups.

For the subset of patients with a genomic score, the concordance index was calculated as a measure of the performance of each method in ranking patients by risk of BCR or metastasis in relation to their actual time to BCR or metastasis, including censored and uncensored outcomes. A concordance index of 0.5 indicates random performance.

For the subset of patients with a genomic score, univariate analysis using Cox proportional-hazards modeling was conducted for each method. Hazard ratios with confidence intervals and p values for the statistical significance of the Wald test are provided for each independent covariate for BCR and for metastasis. The Wald test assesses the ratio of the covariate coefficient to its standard error and evaluates whether the coefficient for a given variable is significantly different from 0. Multivariate analysis was also conducted for all covariates in a single model. Statistical significance using the Wald test was assessed for each covariate. Three global statistical tests were used to measure the overall significance of the multivariate model: the likelihood ratio test, the log-rank test, and the Wald test. These three methods are asymptotically equivalent. As the sample size increases, they all perform similarly. The likelihood ratio test has better performance for smaller sample sizes.

The genomic test score was divided into non-high and high categories using the published threshold value of 0.6. CAPRA-S was divided into low and high categories according to its point system. Because the Kattan and Partin nomograms use continuous scoring systems without designated categorical risk groups, a median threshold was used to define low and high risk categories for comparison of hazard ratios with the other methods.

The net benefit of PATHOMIQ_PRAD in comparison to a genomic score and CAPRA-S was assessed via decision curve analysis (DCA) for the 5-yr probability of BCR and metastasis. The 3-yr probability of BCR was also assessed because of recent clinical interest in identifying risk at an earlier time point. Net benefit was measured based on a range of threshold probabilities to indicate the minimum probability of disease at which an additional intervention would be justified. The net benefit was calculated as sensitivity prevalence-(1−specificity)×(1−prevalence)×w, where w represents the odds at the threshold probability [6]. Individual survival models were trained and used for DCA.

### Model Interpretation

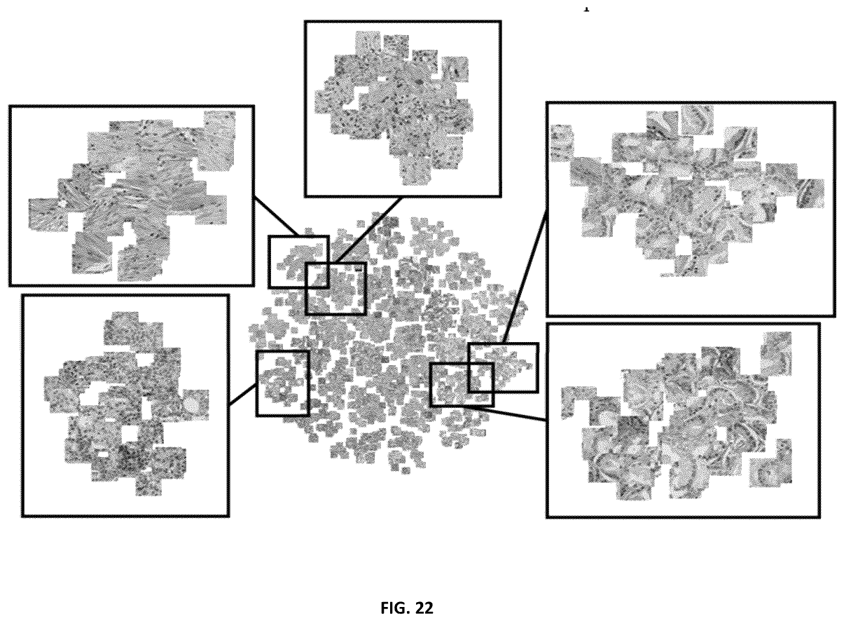

PATHOMIQ_PRAD analyzes both cancer epithelium and the proximal tumor microenvironment (TME). To demonstrate its ability to recognize subtle differences in histological groups, regions of interest (ROIs) correlated most closely with disease progression in the training data were clustered on the basis of image similarity. FIG. 22 shows a high-level view of this cluster map, with some randomly selected morphological clusters highlighted as examples. The center of the figure shows a high-level cluster map of various prostate cancer morphologies learned by PATHOMIQ_PRAD. Boxes show magnified images of some randomly selected examples of morphological sub patterns across the cancer epithelium and stroma and the tumor microenvironment.

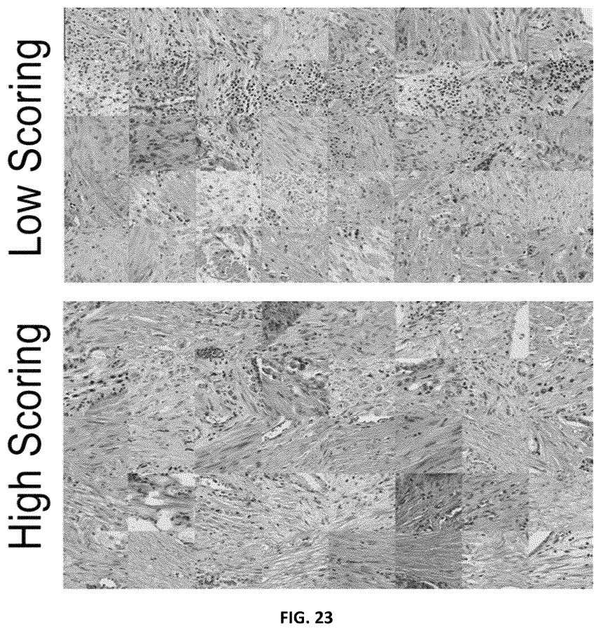

To investigate which features in the TME are most relevant when predicting disease outcome, regions indicative of high risk and low risk are extracted separately from the test data for review by a pathologist and identification of specific stromal features that have previously been largely ignored because of a lack of evidence regarding their role in tumor risk stratification (see FIG. 23). FIG. 23 illustrates examples of high- and low-scoring morphological areas of the tumor microenvironment according to PATHOMIQ_PRAD. The low-scoring stroma has a relatively higher percentage of smooth muscle fibers and lymphocytic infiltrates than the high-scoring stroma, which is composed of mostly reactive fibroblasts/myofibroblasts.

Patients with intermediate-risk prostate cancer (PCa) exhibit a wide range of disease characteristics and clinical outcomes, making their management challenging. Several stratification methods have been developed to better categorize these patients. Effective stratification is crucial for guiding selection of the most appropriate treatment options and improving clinical outcomes for individuals with intermediate-risk PCa.

The D'Amico classification, one of the earliest and most consistently used tools, stratifies PCa patients into risk groups according to their 5-yr BCR risk after radiotherapy or RP. The scheme uses clinical stage, PSA levels, and Gleason score for stratification. Despite its foundational role, there are limitations in relying solely on Gleason grading or other clinical parameters for effective patient stratification. This study highlights how our AI-powered platform addresses these limitations and offers a more precise stratification method for intermediate-risk PCa, thereby enhancing clinical decision-making for better outcomes.

PATHOMQ_PRAD uses digitized hematoxylin and eosin (HE)-stained WSIs and applies a holistic approach that incorporates epithelial, stromal, and immune contexture to generate high-dimensional vectors for each image. This allows numerical representations of the morphologies observed, which are then transformed into a single prognostic score (ranging from 0 of 1) for each patient that is capable of predicting clinical outcomes.

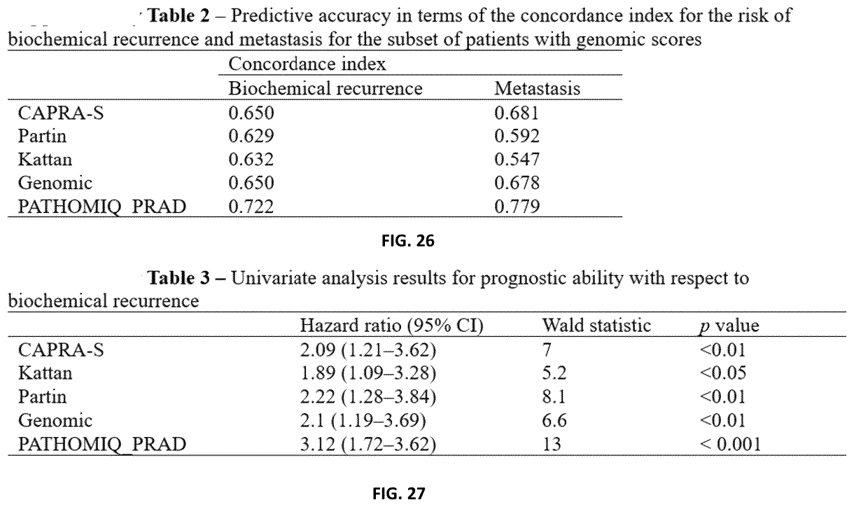

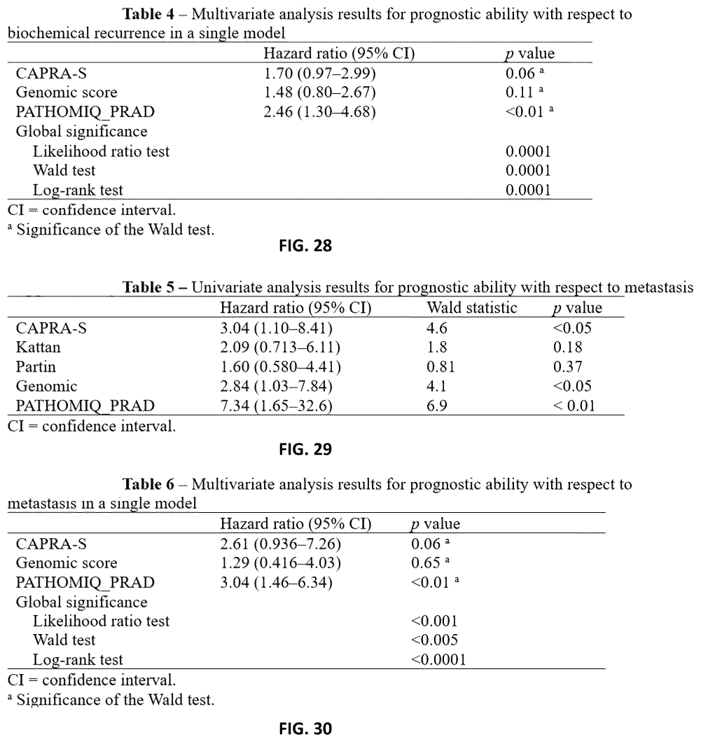

The results in FIG. 20 illustrate the high statistical significance of our PATHOMIQ_PRAD system in stratifying patients with intermediate-risk PCa. PATHOMIQ_PRAD also shows stronger risk stratification for intermediate-risk PCa cases in comparison to nomograms and genomic tests in current clinical use. To assess the net benefit to clinicians, decision curve analysis (DCA) was performed for the 5-yr probability of BCR and metastasis. In both univariate and multivariate analyses for BCR and metastasis, PATHOMIQ_PRAD demonstrated superior performance to other algorithms and scores (Tables 1-5 as depicted in FIGS. 25-27). The DCA analysis for 3-yr and 5-yr BCR risk and for the 5-yr probability of metastasis (See FIG. 20, Table B) confirmed that PATHOMIQ_PRAD provides a superior net benefit in comparison to current nomograms and genomic scores. This underscores the effectiveness of the AI-enabled deep learning technique in analyzing the intricate morphological details of HE-stained slides, revealing critical pathological features that remain undetected by even experienced genitourinary pathologists and are not accounted for in the current GG system.

While we did not evaluate the impact of exclusion of some of these features from the calculation on the predictive accuracy of PATHOMIQ_PRAD, a previous study evaluated ROIs in WSIs with high morphometric scores. Results from a pilot morphogenomic study, which used a NanoString panel of limited protein markers, allowed us to identify factors in the tumor microenvironment that drive tumor growth. This supports the validity of our approach. We discovered that well-known markers of cancer proliferation, such as Ki67, were elevated in these ROIs and reported for the first time that immune markers such as TMEM173, CD8, CD163, and PD-L1 were also highly expressed in the ROIs. This implies that the extensive perspective offered by PATHOMIQ_PRAD might encompass predictive characteristics that have not yet been identified. In addition, advanced technologies such as spatial transcriptomics using comprehensive whole-transcriptome assays and mass cytometry can further aid in this approach. These strategies are currently being explored in our laboratories.

PATHOMIQ_PRAD has been validated across multiple risk assessment and treatment response studies that highlighted its remarkable generalizability and broad clinical utility for PCa management. While the studies described here used WSIs for RP specimens, we tested the predictive capability of PATHOMIQ_PRAD on biopsy specimens and found that it performs equally well with biopsies. For a cohort of 436 patients, PATHOMIQ_PRAD showed stronger stratification for patients on apalutamide+ADT (hazard ratio 0.19, 95% confidence interval [CI] 0-0.37; p<0.005) versus placebo+ADT (hazard ratio 0.39, 95% CI, 0.17-0.86; p=0.02).

Our prior research demonstrated the high accuracy of the test in predicting BCR across all PCa risk categories, now expanded to include precise predictions of both BCR and metastasis for patients with intermediate-risk PCa. This novel AI platform can potentially fill crucial gaps in treatment decision-making for intermediate-risk PCa, particularly in evaluating the need for RP versus watchful waiting and in identifying patients who may benefit from more aggressive treatments, such as ADT and androgen receptor signaling inhibitors (ARSIs), after surgery. In addition, this approach has a fast turnaround time and lower costs, involves decentralized deployment, and does not destroy tissue, which are advantages in comparison to genomic tests.

In summary, our results to date show that the PATHOMIQ_PRAD test can guide clinical decision-making and is potentially ready for clinical translation and incorporation into routine practice, pending appropriate regulatory approvals. PATHOMIQ_PRAD is the first AI-driven prognostic tool tailored for intermediate-risk PCa after RP that can discern patients likely to respond to ADT or ARSI therapy to prevent or slow disease progression.

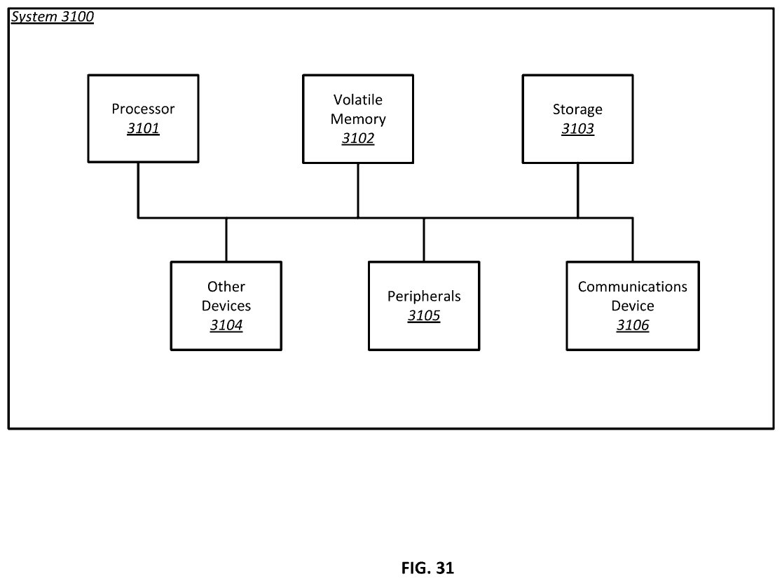

FIG. 31 is a diagram illustrating an exemplary computer that may perform processing in some embodiments. Exemplary computer 3100 may perform operations consistent with some embodiments. The architecture of computer 3100 is exemplary. Computers can be implemented in a variety of other ways. A wide variety of computers can be used in accordance with the embodiments herein.

Processor 3101 may perform computing functions such as running computer programs. The volatile memory 3102 may provide temporary storage of data for the processor 3101. RAM is one kind of volatile memory. Volatile memory typically requires power to maintain its stored information. Storage 3103 provides computer storage for data, instructions, and/or arbitrary information. Non-volatile memory, which can preserve data even when not powered and including disks and flash memory, is an example of storage. Storage 3103 may be organized as a file system, database, or in other ways. Data, instructions, and information may be loaded from storage 3103 into volatile memory 3102 for processing by the processor 3101.

The computer 3100 may include peripherals 3105. Peripherals 3105 may include input peripherals such as a keyboard, mouse, trackball, video camera, microphone, and other input devices. Peripherals 3105 may also include output devices such as a display. Peripherals 3105 may include removable media devices such as CD-R and DVD-R recorders/players. Communications device 3106 may connect the computer 3100 to an external medium. For example, communications device 3106 may take the form of a network adapter that provides communications to a network. A computer 3100 may also include a variety of other devices 3104. The various components of the computer 3100 may be connected by a connection medium such as a bus, crossbar, or network.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any programming language. It will be appreciated that a variety of programming languages may be used to implement the teachings of the disclosure as described herein.

What is claimed is:

1. A method for identifying prostate cancer patients at high-risk of progression among clinically intermediate risk group, comprising:
obtaining images of patient cells;
tiling the images into subsets of smaller images;
performing a morphology quantification process on the subsets of smaller images, comprising:
inputting a portion of the images into one or more trained machine learning models;
determining by the one or more trained machine learning models likely cancer cells and a grading of the cancer cells; and
identifying whether the cells in the subsets of smaller images indicate whether a prostate cancer patient is at a risk of progression among a clinically intermediate risk group.

2. The method of claim 1, wherein the grading of the cancer cells classifies cells as GP3, GP5 and/or necrotic cells.

3. The method of claim 1, wherein the trained machine learning model determines whether patient cells are cancerous cells or benign stroma.

4. The method of claim 1, further wherein the one or more trained machine learning networks are trained on multiple images of cancer cells and benign stroma.

5. The method of claim 1, further comprising:
substratifying GGG2 patients and GGG3 patients with high statistical significance.

6. The method of claim 1, further comprising:
performing a feature encoding process on the subsets of smaller images.

7. The method of claim 6, wherein the feature encoding process, comprises:
performing semi-supervised encoding;
performing patient level aggregation; and
performing dimensionality reduction.

8. The method of claim 1, wherein tiling the images comprises:
dividing a first set of patient tissue image slides into a plurality of tiles of a predetermined pixel size.

9. The method of claim 1, further comprising the operations of:
removing tiles of the subset of smaller images that have any pen marks, scanning artifacts, tissue folds or other degrading characteristics in the tile.

10. The method of claim 7, further comprising the operations of:
combining an output of the morphology quantification process, and an output of the feature encoding process as covariants; and
inputting the covariant into a final survival model which is trained to output a score between 0 and 1, with the score indicating a risk of biochemical recurrence, with 1 being the highest likelihood of recurrence.

11. A system for identifying prostate cancer patients at high-risk of progression among clinically intermediate risk group, the system comprising one or more processors configured to perform the operations of:
obtaining images of patient cells;
tiling the images into subsets of smaller images;
performing a morphology quantification process on the subsets of smaller images, comprising:
inputting a portion of the images into one or more trained machine learning models;
determining by the one or more trained machine learning models likely cancer cells and a grading of the cancer cells; and
identifying whether the cells in the subsets of smaller images indicate whether a prostate cancer patient is at a risk of progression among a clinically intermediate risk group.

12. The system of claim 11, wherein the grading of the cancer cells classifies cells as GP3, GP5 and/or necrotic cells.

13. The system of claim 11, wherein the trained machine learning model determines whether patient cells are cancerous cells or benign stroma.

14. The system of claim 11, further wherein the one or more trained machine learning networks are trained on multiple images of cancer cells and benign stroma.

15. The system of claim 11, further comprising the operations of:
substratifying GGG2 patients and GGG3 patients with high statistical significance.

16. The system of claim 11, further comprising the operations of:
performing a feature encoding process on the subsets of smaller images.

17. The system of claim 16, wherein the feature encoding process, comprises:
performing semi-supervised encoding;
performing patient level aggregation; and
performing dimensionality reduction.

18. The system of claim 11, wherein tiling the images comprises:
dividing a first set of patient tissue image slides into a plurality of tiles of a predetermined pixel size.

19. The system of claim 11, further comprising the operations of:
removing tiles of the subset of smaller images that have any pen marks, scanning artifacts, tissue folds or other degrading characteristics in the tile.

20. The method of claim 7, further comprising the operations of:
combining an output of the morphology quantification process, and an output of the feature encoding process as covariants; and
inputting the covariant into a final survival model which is trained to output a score between 0 and 1, with the score indicating a risk of biochemical recurrence, with 1 being the highest likelihood of recurrence.
